(19) United States
(12) Patent Application Publication
(10) Pub. No.: US 2020/0397817 A1
(43) Pub. Date: Dec. 24, 2020

(54) METHOD FOR PREDICTING AND MODULATING SUSCEPTIBILITY OF CANCER CELL TO PROGRAMMED CELL DEATH

(71) Applicant: SHIEH, Darbin, Tainan (TW)

(72) Inventors: DAR-BIN SHIEH, Tainan (TW); KUANG-JING HUANG, Tainan (TW); LI-XING YANG, New Taipei (TW)

(21) Appl. No.: 16/767,121

(22) PCT Filed: Nov. 30, 2017
(86) PCT No.: PCT/CN2017/113796

§ 371 Date: May 27, 2020

(51) Int. Cl. A61K 33/26; A61K 33/242; C12Q 1/6886

(52) U.S. Cl. CPC A61K 33/26; C12Q 2600/106; C12Q 1/6886; A61K 33/242

(57) ABSTRACT

A method for treating cancer includes the steps of: administering to a subject an effective amount of zero valent iron (ZVI) nanoparticles and an effective amount of at least one resistance modulating agent. The shell of each of the ZVI nanoparticles includes gold (Au). The resistance modulating agent includes inducers of programmed cell death, such as erastin, sulfasalazine, sorafenib, buthionine sulfoximine, Ras selective lethal 3 (RSL-3), altretamine, and FIN56. The resistance modulating agent can induce ferroptosis, promote lipid peroxidation, block NADP(H) supply, or suppress metabolism of polyunsaturated fatty acids. Alternatively, the resistance modulating agent can suppress expression of at least one of GSR, AKR1C1, AKR1C3, AKR1B1, AKR1B10 and KYNU genes or promote expression of at least one of ACSL4, ZEB1 and NNMT genes. A method for improving efficacy of a cancer treatment using the ZVI nanoparticles and another method for treating cancer are also provided.

### FIELD OF THE INVENTION

The present disclosure relates to a method for treating cancer, and more particularly to methods for modulating and predicting susceptibility of cancer cells to a non-apoptotic cell death pathway.

### BACKGROUND OF THE INVENTION

Over the past decade, the application of nanoparticles to the field medicine has opened new routes for drug formulation and multimodal therapies. Conventionally, nanoparticles have served majorly as drug carriers or relays for externally applied energy (eg. applying a gradient of magnetic field to injectable magnetizable nanoparticles that are distributed to surround a tumor as disclosed in US20160143859). Not until only recently, nanoparticles have started to be explored for their intrinsic anti-cancer activities in blocking signaling pathways associated with metastasis, mitochondrial biogenesis, and escaping from cell death.

For example, nanoparticles having zero valent irons at the cores thereof, also known as zero valent iron nanoparticles (ZVI NPs), have been shown in US20130236548 to selectively kill cancer cells over normal cells and inhibit tumor growth, when administered alone without carrying any drug. However, application of ZVI NPs in treating cancer has been limited, as a significant percentage of cancers have shown to develop resistance to ZVI NP treatment. For example, as shown in FIG. 1, oral squamous cell carcinoma (OSCC) cancer cell lines HSC-3, SAS, KOSC-3, and OC2 were shown to be refractory to treatment with various types of ZVI NPs, including bare ZVI NPs, ZVI NPs with gold shells (denoted as ZVI@Au NPs), carboxymethyl cellulose (CMC) stabilized ZVI NPs (denoted ZVI@CMC NPs) and CMC stabilized ZVI NPs with gold shells (denoted CMC stabilized ZVI@Au NPs), whereas other OSCC cell lines, such as OC3, OEC-M1, and SCC-9 cells, were shown to be ZVI sensitive under the same conditions.

Therefore, there is a need for a method for modulating susceptibility and overcoming resistance of cancer cells to ZVI NP treatments. There is also a need for a method for predicting efficacy of ZVI NP treatments.

### BRIEF SUMMARY OF THE INVENTION

An aspect of the present invention provides a method for treating cancer. The method includes the steps of: administering to a subject an effective amount of zero valent iron (ZVI) nanoparticles and an effective amount of at least one resistance modulating agent.

In a preferred embodiment, the shell of each of the ZVI nanoparticles comprises gold (Au).

In a preferred embodiment, the resistance modulating agent induces ferroptosis, promotes lipid peroxidation, blocks NADP(H) supply, or suppresses metabolism of polyunsaturated fatty acids.

In a preferred embodiment, the resistance modulating agent suppresses expression of at least one of GSR, AKR1C1, AKR1C3, AKR1B1, AKR1B10, and KYNU genes or promotes expression of at least one of ACSL4, ZEB1 and NNMT genes.

In a preferred embodiment, the resistance modulating agent is selected from a group consisting of small molecules, peptides, proteins, nucleotides, nanoparticles, and metal-based nanostructures.

In a preferred embodiment, the small molecule comprises erastin, sulfasalazine, sorafenib, buthionine sulfoximine, Ras selective lethal 3 (RSL-3), altretamine, and FIN56.

In a preferred embodiment, the cancer to be treated by the method includes oral cancer, head and neck cancer, bladder cancer, bone cancer, brain cancer, breast cancer, cervical cancer, colon cancer, endometrial cancer, esophageal cancer, leukemia, liver cancer, lymphoma, kidney cancer, osteosarcoma, ovarian cancer, pancreatic cancer, prostate cancer, skin cancer, small intestine cancer, stomach cancer, thymus cancer and thyroid cancer.

Another aspect of the present invention provides a method for improving efficacy of a cancer treatment. The method includes the steps of: administering an effective amount of at least one of the aforementioned resistance modulating agent to a subject receiving a treatment using the aforementioned ZVI nanoparticles.

In a preferred embodiment, the cancer treated by the cancer treatment includes oral cancer, head and neck cancer, bladder cancer, bone cancer, brain cancer, breast cancer, cervical cancer, colon cancer, endometrial cancer, esophageal cancer, leukemia, liver cancer, lymphoma, kidney cancer, osteosarcoma, ovarian cancer, pancreatic cancer, prostate cancer, skin cancer, small intestine cancer, stomach cancer, thymus cancer and thyroid cancer.

Yet another aspect of the present invention provides another method for treating cancer, comprising steps of: obtaining a first transcriptome profile of cancer cells of a subject; providing the cancer cells a prophylactically effective amount of the aforementioned ZVI nanoparticles; obtaining a second transcriptome profile of the cancer cells; determining susceptibility of the cancer cells to the ZVI nanoparticles according to the difference between the first and second transcriptome profiles; and treating the subject according to a result of the determination.

In a preferred embodiment, the first and second transcriptome profiles includes expression levels of at least one of GSR, AKR1C1, AKR1C3, AKR1B1, AKR1B10, KYNU, ACSL4, ZEB1 and NNMT genes.

In a preferred embodiment, if the cancer cells are determined to be susceptible to the ZVI nanoparticles, the subject is treated by administering to the subject a therapeutically effective amount of ZVI nanoparticles.

In a preferred embodiment, if the cancer cells are determined to be not susceptible to the ZVI nanoparticles, the subject is treated by administering to the subject a therapeutically effective amount of ZVI nanoparticles and a therapeutically effective amount of at least one of the aforementioned resistance modulating agent.

In a preferred embodiment, the cancer to be treated by the method includes oral cancer, head and neck cancer, bladder cancer, bone cancer, brain cancer, breast cancer, cervical cancer, colon cancer, endometrial cancer, esophageal cancer, leukemia, liver cancer, lymphoma, kidney cancer, osteosarcoma, ovarian cancer, pancreatic cancer, prostate cancer, skin cancer, small intestine cancer, stomach cancer, thymus cancer and thyroid cancer.

In sum, the methods of using zero valent iron (ZVI) nanoparticles in combination with ferroptosis inducers according to the various embodiments of the present invention provide synergistic therapeutic effect and enhance efficacy of ZVI NP treatments by modulating susceptibility and overcoming resistance of cancer cells to ZVI without affecting normal cells or causing undesirable side effects. Meanwhile, other embodiments of the present invention also reveal the key genetic markers for predicting efficacy of ZVI NP treatment. Therefore, the present invention offers new strategies for treating cancer and improving clinical outcome of nanomedicine.

### BRIEF DESCRIPTION OF THE DRAWINGS

The accompanying drawings illustrate one or more embodiments of the present invention and, together with the written description, explain the principles of the present invention. Wherever possible, the same reference numbers are used throughout the drawings to refer to the same or like elements of an embodiment.

FIG. 1 is an experimental result showing resistance of cancer cells to ZVI NP treatment (ie. ZVI resistance) in accordance with the prior art;

FIG. 2A is an experimental result showing the correlation between ZVI resistance and production of hydrogen peroxide (H₂O₂) in accordance with an embodiment of the present invention;

FIG. 2B is an experimental result showing the correlation between ZVI resistance and production of intracellular reactive oxygen species (ROS) in accordance with an embodiment of the present invention;

FIG. 2C is an experimental result showing the correlation between ZVI resistance and production of mitochondrial ROS in accordance with an embodiment of the present invention;

FIG. 3 is an experimental result showing the association between ZVI resistance and presence of ferrous iron (Fe²⁺) in accordance with an embodiment of the present invention;

FIG. 4 is an experimental result showing the association between ZVI resistance and production of intracellular ROS in accordance with an embodiment of the present invention;

FIG. 5A is an experimental result showing a non-apoptotic cell death induced by ZVI NPs in accordance with an embodiment of the present invention;

FIG. 5B is an experimental result showing the association between mitochondria dysfunction and cell death induced by ZVI NPs in accordance with an embodiment of the present invention;

FIG. 6A is an experimental result showing the generation of ZVI refractory cell clones from a ZVI sensitive cell clone in accordance with an embodiment of the present invention;

FIG. 6B, FIG. 6C and FIG. 6D are experimental results showing the association between ZVI resistance and mitochondrial hyperoxidation in accordance with an embodiment of the present invention;

FIG. 7A is an experimental result showing the association between ZVI resistance and lipid peroxidation in accordance with an embodiment of the present invention;

FIG. 7B is an experimental result showing the association between ZVI resistance and ferroptosis in accordance with an embodiment of the present invention;

FIG. 7C is another experimental result showing the association between ZVI resistance and ferroptosis in accordance with an embodiment of the present invention;

FIG. 8 is an experimental result showing the association between ZVI resistance and expression of glutathione peroxidases (GPx) in accordance with an embodiment of the present invention;

FIG. 9A is an experimental results showing transcriptome profile of ZVI sensitive and resistance cells in accordance with an embodiment of the present invention;

FIG. 9B is the proposed biosynthetic pathways associated with ferroptosis in accordance with an embodiment of the present invention;

FIG. 10A is the in vitro experimental results showing the synergistic effect of ferroptosis inducers in ZVI NP treatments in accordance with an embodiment of the present invention; and

FIG. 10B, FIG. 10C, FIG. 10D are the in vivo experimental results showing the synergistic therapeutic effect of ferroptosis inducers in ZVI NP treatments in accordance with an embodiment of the present invention.

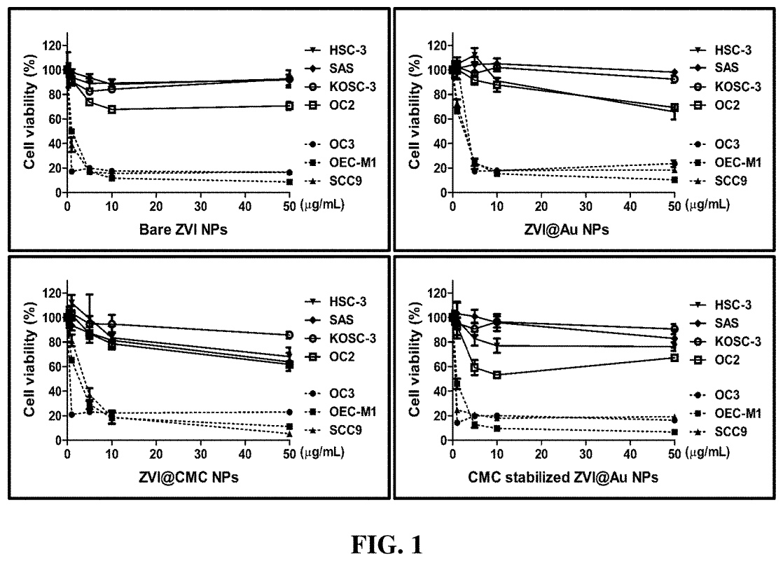

In accordance with common practice, the various described features are not drawn to scale and are drawn to emphasize features relevant to the present disclosure. Like reference characters denote like elements throughout the figures and text.

### DETAILED DESCRIPTION OF THE PREFERRED EMBODIMENTS

The present invention will now be described more fully hereinafter with reference to the accompanying drawings illustrating various exemplary embodiments of the invention. The present invention may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

It will be understood that the term “and/or” includes any and all combinations of one or more of the associated listed items. It will also be understood that, although the terms first, second, third etc. may be used herein to describe various elements, components, regions, parts and/or sections, these elements, components, regions, parts and/or sections should not be limited by these terms. These terms are only used to distinguish one element, component, region, part or section from another element, component, region, layer or section. Thus, a first element, component, region, part or section discussed below could be termed a second element, component, region, layer or section without departing from the teachings of the present disclosure.

The term “treating” or “treatment” used in the present disclosure encompasses both disease-modifying treatment and symptomatic treatment, either of which may be prophylactic (i.e., before the onset of symptoms, in order to prevent, delay or reduce the severity of symptoms, or to induce resistance against the treatment) or therapeutic (i.e., after the onset of symptoms, in order to reduce the severity and/or duration of symptoms). Treatment methods provided herein include, in general, administration to a subject an effective amount of one or more small molecules, peptides, antibodies, RNAi, or aptamers provided herein. Suitable subjects include patients suffering from or susceptible to a disorder or disease identified herein. Typical subjects for treatment as described herein include mammals, particularly primates, especially humans. Other suitable subjects include domesticated companion animals, such as a dog, cat, horse, and the like, or a livestock animal such as cattle, pig, sheep and the like.

The “cancer” described herein includes any types of cancer generally known in the art, such as oral cancer, head and neck cancer, bladder cancer, bone cancer, brain cancer, breast cancer, cervical cancer, colon cancer, endometrial cancer, esophageal cancer, leukemia, liver cancer, lymphoma, kidney cancer, osteosarcoma, ovarian cancer, pancreatic cancer, prostate cancer, skin cancer including basal and squamous cell carcinoma and melanoma, small intestine cancer, stomach cancer, thymus cancer and thyroid cancer. Preferably, the methods provided herein according by various embodiments of the present disclosure is used to treat various histological types of oral cancer, such as teratoma, adenocarcinoma derived from a major or minor salivary gland, lymphoma from tonsillar or other lymphoid tissue, or melanoma from pigment-producing cells of the oral mucosa.

An “effective amount” used herein includes a “therapeutically effective amount” and a “prophylactically effective amount.” A “therapeutically effective amount” refers to an amount effective, at dosages and for periods of time necessary, to achieve the desired therapeutic result, such as suppression or inhibition of tumor growth. The therapeutically effective amount may vary according to disease state, age, gender, and weight of the subject, route of administration, ability of the active ingredient(s) to elicit a desired response in the subject, and use of excipient(s) or with other active ingredients. Dosage regimens may be adjusted to provide the optimum therapeutic response. A therapeutically effective amount is also one in which any toxic or detrimental effects are outweighed by the therapeutically beneficial effects.

A “prophylactically effective amount” refers to an amount effective, at dosages and for periods of time necessary, to achieve the desired prophylactic result, such as preventing or inhibiting metastasis of a tumor, or to induce resistance against the treatment. A prophylactically effective amount can be determined as described above for the therapeutically effective amount. Typically, since a prophylactic dose is used in subjects prior to or at an earlier stage of disease, the prophylactically effective amount shall be less than the therapeutically effective amount.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

An aspect of the present invention provides a method for treating cancer. In an embodiment, the method includes the step of: administering to a subject suffering from or susceptible to a cancer an effective amount of zero valent iron (ZVI) nanoparticles and an effective amount of at least one resistance modulating agent. The core of each of the ZVI nanoparticles (hereinafter ZVI NPs) includes a zero valent iron. The shell of each of the ZVI NPs may partially or entirely cover the ZVI core and include a metal (preferably gold (Au)), a metal doped with dopants, a metal alloy, a polymer, carbon, a metal oxide or a nonmetal oxide. Preferably, the thickness of the shell ranges from about 0.7 nm to about 6 nm. The ZVI NPs may further be stabilized with carboxy methyl cellulose (CMC), an Au layer or other nanoparticle stabilizers. The ZVI NPs may be rod-shaped, spheric, cubic or dumbbell-shaped. Particle size of each of the ZVI NPs ranges between about 5 nm to about 5 μm, preferably 5 nm to 1 μm, or more preferably 5 nm to 50 nm.

The resistance modulating agent may include small molecules, peptides, proteins, nucleotides, nanoparticles, and/or metal-based nanostructures that can induce ferroptosis, promote lipid peroxidation, block supply of nicotinamide adenine dinucleotide/nicotinamide adenine dinucleotide phosphate (NADP(H)), or suppress metabolism of polyunsaturated fatty acids. For example, the small molecule may be erastin (2-[1-[4-[2-(4-chlorophenoxy)acetyl]piperazin-1-yl]ethyl]-3-(2-ethoxyphenyl)quinazolin-4-one), sulfasalazine ((3Z)-6-oxo-3-[[4-(pyridin-2-ylsulfamoyl)phenyl]hydrazinylidene]cyclohexa-1,4-diene-1-carboxylic acid), sorafenib (4-[4-[[4-chloro-3-(trifluoromethyl)phenyl]carbamoylamino]phenoxy]-N-methylpyridine-2-carboxamide), buthionine sulfoximine (2-amino-4-(butylsulfonimidoyl)butanoic acid), Ras selective lethal 3 (RSL-3; methyl (1S,3R)-2-(2-chloroacetyl)-1-(4-methoxycarbonylphenyl)-1,3,4,9-tetrahydropyrido[3,4-b]indole-3-carboxylate), altretamine (2-N,2-N,4-N,4-N,6-N,6-N-hexamethyl-1,3,5-triazine-2,4,6-triamine), FIN56 (2-N,7-N-dicyclohexyl-9-hydroxyiminofluorene-2,7-disulfonamide), or other ferroptosis inducers (FINs) or lipid peroxidation inhibitors (LPOi). Non-small molecules that have been shown to induce ferroptosis includes silica-based nanoparticles Cornell dots (also known as C dots) and a nano-sized metal-organic network encapsulating a p53 plasmid.

The resistance modulating agent can suppress or downregulate expression of at least one of the following genes, as identified by Entrez Gene IDs according to the NCBI gene database (www.ncbi.nlm.nih.gov/entrez/query.fcgi?db=gene): AATK (Entrez Gene ID: 9625), ABAT (Entrez Gene ID: 18), ABCA10 (Entrez Gene ID: 10349), ABHD8 (Entrez Gene ID: 79575), ABTB2 (Entrez Gene ID: 25841), ACADVL (Entrez Gene ID: 37), ACBD4 (Entrez Gene ID: 79777), ACBD7 (Entrez Gene ID: 414149), ACO1 (Entrez Gene ID: 48), ACOT4 (Entrez Gene ID: 122970), ADAMTS6 (Entrez Gene ID: 11174), ADGRF4 (Entrez Gene ID: 221393), ADGRV1 (Entrez Gene ID: 84059), AGR2 (Entrez Gene ID: 10551), AIFM2 (Entrez Gene ID: 84883), AIM1 (Entrez Gene ID: 202), AKAP12 (Entrez Gene ID: 9590), AKR1B1 (Entrez Gene ID: 231), AKR1B10 (Entrez Gene ID: 57016), AKR1C1 (Entrez Gene ID: 1645), AKR1C3 (Entrez Gene ID: 8644), AKR1C4 (Entrez Gene ID: 1109), ALPK1 (Entrez Gene ID: 80216), ALPK3 (Entrez Gene ID: 57538), AMN (Entrez Gene ID: 81693), AMPD3 (Entrez Gene ID: 272), ANKRD22 (Entrez Gene ID: 118932), ANKRD65 (Entrez Gene ID: 441869), ANXA8 (Entrez Gene ID: 653145), ANXA8L1 (Entrez Gene ID: 728113), AOAH (Entrez Gene ID: 313), APOBEC3F (Entrez Gene ID: 200316), APOL1 (Entrez Gene ID: 8542), APOL6 (Entrez Gene ID: 80830), AQP3 (Entrez Gene ID: 360), AREG (Entrez Gene ID: 374), ARHGAP4 (Entrez Gene ID: 393), ARHGEF4 (Entrez Gene ID: 50649), ARHGEF5 (Entrez Gene ID: 7984), ARL14 (Entrez Gene ID: 80117), ASAH1 (Entrez Gene ID: 427), ATP6V0A4 (Entrez Gene ID: 50617), B3GALNT1 (Entrez Gene ID: 8706), BAIT (Entrez Gene ID: 10538), BBC3 (Entrez Gene ID: 27113), BIK (Entrez Gene ID: 638), BIRC3 (Entrez Gene ID: 330), BMF (Entrez Gene ID: 90427), BNIP3L (Entrez Gene ID: 665), BTN3A1 (Entrez Gene ID: 11119), C10orf35 (Entrez Gene ID: 219738), C11orf86 (Entrez Gene ID: 254439), C12orf75 (Entrez Gene ID: 387882), C15orf56 (Entrez Gene ID: 644809), C1orf116 (Entrez Gene ID: 79098), C1QTNF1-AS1 (Entrez Gene ID: 100507410), CTRB1 (Entrez Gene ID: 1504), C3 (Entrez Gene ID: 718), C4orf19 (Entrez Gene ID: 55286), C4orf26 (Entrez Gene ID: 152816), C8orf4 (Entrez Gene ID: 56892), CA2 (Entrez Gene ID: 760), CABYR (Entrez Gene ID: 26256), CALHM2 (Entrez Gene ID: 51063), CALHM3 (Entrez Gene ID: 119395), CAMK2G (Entrez Gene ID: 818), CAPG (Entrez Gene ID: 822), CAPS (Entrez Gene ID: 828), CARD6 (Entrez Gene ID: 84674), CARDS (Entrez Gene ID: 64170), CARF (Entrez Gene ID: 79800), CARHSP1 (Entrez Gene ID: 23589), CASK (Entrez Gene ID: 8573), CASP10 (Entrez Gene ID: 843), CASZ1 (Entrez Gene ID: 54897), CBLC (Entrez Gene ID: 23624), CCL20 (Entrez Gene ID: 6364), CCNG2 (Entrez Gene ID: 901), CD24 (Entrez Gene ID: 100133941), CD9 (Entrez Gene ID: 928), CDA (Entrez Gene ID: 978), CDH1 (Entrez Gene ID: 999), CDKN2B (Entrez Gene ID: 1030), CEBPD (Entrez Gene ID: 1052), CFB (Entrez Gene ID: 629), CHRNB1 (Entrez Gene ID: 1140), CHST15 (Entrez Gene ID: 51363), CLDN1 (Entrez Gene ID: 9076), COCH (Entrez Gene ID: 1690), COL9A3 (Entrez Gene ID: 1299), CORO6 (Entrez Gene ID: 84940), CPEB2 (Entrez Gene ID: 132864), CPVL (Entrez Gene ID: 54504), CRACR2B (Entrez Gene ID: 283229), CRIP1 (Entrez Gene ID: 1396), CSGALNACT1 (Entrez Gene ID: 55790), CTRB2 (Entrez Gene ID: 440387), CTSD (Entrez Gene ID: 1509), CTSS (Entrez Gene ID: 1520), CTSV (Entrez Gene ID: 1515), CXCL1 (Entrez Gene ID: 2919), CXCL10 (Entrez Gene ID: 3627), CXCL16 (Entrez Gene ID: 58191), CXCL2 (Entrez Gene ID: 2920), CXCL8 (Entrez Gene ID: 3576), DAPP1 (Entrez Gene ID: 27071), DBN1 (Entrez Gene ID: 1627), DCLRE1C (Entrez Gene ID: 64421), DDAH2 (Entrez Gene ID: 23564), DDTL (Entrez Gene ID: 100037417), DDX58 (Entrez Gene ID: 23586), DIAPH2 (Entrez Gene ID: 1730), DIO2 (Entrez Gene ID: 1734), DNAJB2 (Entrez Gene ID: 3300), DNAJC1 (Entrez Gene ID: 64215), DNER (Entrez Gene ID: 92737), DSP (Entrez Gene ID: 1832), DUSP10 (Entrez Gene ID: 11221), DUSP18 (Entrez Gene ID: 150290), EEF1A2 (Entrez Gene ID: 1917), ELMO3 (Entrez Gene ID: 79767), ENKD1 (Entrez Gene ID: 84080), ESRP1 (Entrez Gene ID: 54845), ESX1 (Entrez Gene ID: 80712), EXOC3-AS1 (Entrez Gene ID: 116349), F11R (Entrez Gene ID: 50848), F2RL1 (Entrez Gene ID: 2150), FABP6 (Entrez Gene ID: 2172), FAM114A1 (Entrez Gene ID: 92689), FAM229A (Entrez Gene ID: 100128071), FAM26F (Entrez Gene ID: 441168), FAM46A (Entrez Gene ID: 55603), FAM47E-STBD1 (Entrez Gene ID: 100631383), FAM50B (Entrez Gene ID: 26240), FAM83A (Entrez Gene ID: 84985), FAM84B (Entrez Gene ID: 157638), FARP2 (Entrez Gene ID: 9855), FBLIM1 (Entrez Gene ID: 54751), FBXO2 (Entrez Gene ID: 26232), FBXO32 (Entrez Gene ID: 114907), FBXO48 (Entrez Gene ID: 554251), FGD6 (Entrez Gene ID: 55785), FGF11 (Entrez Gene ID: 2256), FHOD3 (Entrez Gene ID: 80206), FILIP1L (Entrez Gene ID: 11259), FKBP11 (Entrez Gene ID: 51303), FKBP1A-SDCBP2 (Entrez Gene ID: 100528031), FLRT3 (Entrez Gene ID: 23767), FRAS1 (Entrez Gene ID: 80144), FRAT1 (Entrez Gene ID: 10023), FREM3 (Entrez Gene ID: 166752), FST (Entrez Gene ID: 10468), FUT11 (Entrez Gene ID: 170384), GABARAPL1 (Entrez Gene ID: 23710), GALM (Entrez Gene ID: 130589), GAREM (Entrez Gene ID: 64762), GBP2 (Entrez Gene ID: 2634), GCNT2 (Entrez Gene ID: 2651), GDA (Entrez Gene ID: 9615), GFPT2 (Entrez Gene ID: 9945), GM2A (Entrez Gene ID: 2760), GNA15 (Entrez Gene ID: 2769), GNAO1 (Entrez Gene ID: 2775), GNG4 (Entrez Gene ID: 2786), GPNMB (Entrez Gene ID: 10457), GPR39 (Entrez Gene ID: 2863), GPRCSB (Entrez Gene ID: 51704), GSN (Entrez Gene ID: 2934), GSR (Entrez Gene ID: 2936), GUSBP3 (Entrez Gene ID: 653188), H1FX (Entrez Gene ID: 8971), EIBP1 (Entrez Gene ID: 26959), HDAC9 (Entrez Gene ID: 9734), HENMT1 (Entrez Gene ID: 113802), HMGCS1 (Entrez Gene ID: 3157), HMEA1 (Entrez Gene ID: 23526), HOXD10 (Entrez Gene ID: 3236), HS3ST1 (Entrez Gene ID: 9957), IAH1 (Entrez Gene ID: 285148), IER3 (Entrez Gene ID: 8870), IFNGR1 (Entrez Gene ID: 3459), IGSF3 (Entrez Gene ID: 3321), IL20RB (Entrez Gene ID: 53833), IL22RA1 (Entrez Gene ID: 58985), IL32 (Entrez Gene ID: 9235), IL4I1 (Entrez Gene ID: 259307), IL6 (Entrez Gene ID: 3569), INHBA (Entrez Gene ID: 3624), INHBB (Entrez Gene ID: 3625), IQCE (Entrez Gene ID: 23288), IRF6 (Entrez Gene ID: 3664), IRF7 (Entrez Gene ID: 3665), IRX4 (Entrez Gene ID: 50805), ITGA2 (Entrez Gene ID: 3673), ITGB8 (Entrez Gene ID: 3696), JUP (Entrez Gene ID: 3728), KCNMB4 (Entrez Gene ID: 27345), KCNN4 (Entrez Gene ID: 3783), KCNS1 (Entrez Gene ID: 3787), KCTD11 (Entrez Gene ID: 147040), KDM6A (Entrez Gene ID: 7403), KHDC1L (Entrez Gene ID: 100129128), KIAA1462 (Entrez Gene ID: 57608), KLHDC9 (Entrez Gene ID: 126823), KLHL24 (Entrez Gene ID: 54800), KLRC1 (Entrez Gene ID: 3821), KLRC2 (Entrez Gene ID: 3822), KLRC3 (Entrez Gene ID: 3823), KMO (Entrez Gene ID: 8564), KRT81 Entrez Gene ID: 3887), KRTAP4-1 (Entrez Gene ID: 85285), KYNU (Entrez Gene ID: 8942), LAMA3 (Entrez Gene ID: 3909), LAMA4 (Entrez Gene ID: 3910), LAMB3 (Entrez Gene ID: 3914), LAMC2 (Entrez Gene ID: 3918), LCN2 (Entrez Gene ID: 3934), LCP1 (Entrez Gene ID: 3936), LINC00525 (Entrez Gene ID: 84847), LINC00623 (Entrez Gene ID: 728855), LINC00847 (Entrez Gene ID: 729678), LINC01137 (Entrez Gene ID: 728431), LMBRD1 (Entrez Gene ID: 55788), LOC100128242 (Entrez Gene ID: 100128242), LOC100287290 (Entrez Gene ID: 100287290), LOC154761 (Entrez Gene ID: 154761), LOC644189 (Entrez Gene ID: 644189), LOC728392 (Entrez Gene ID: 728392), LRG1 (Entrez Gene ID: 116844), LTB (Entrez Gene ID: 4050), LTBP2 (Entrez Gene ID: 4053), LYPD3 (Entrez Gene ID:27076), LYPD6 (Entrez Gene ID: 130574), MAGED2 (Entrez Gene ID: 10916), MAL2 (Entrez Gene ID: 114569), MAN1A1 (Entrez Gene ID:4121), MAP3K5 (Entrez Gene ID: 4217), MAP3K8 (Entrez Gene ID: 1326), MARCKSL1 (Entrez Gene ID: 65108), MARVELD3 (Entrez Gene ID: 91862), MB (Entrez Gene ID: 4151), MCAM (Entrez Gene ID: 4162), MEX3A (Entrez Gene ID: 92312), MFI2 (Entrez Gene ID: 4241), MGAM (Entrez Gene ID: 8972), MGAT4A (Entrez Gene ID: 11320), MGP (Entrez Gene ID: 4256), MIR205HG (Entrez Gene ID: 642587), MLF1 (Entrez Gene ID: 4291), MMD (Entrez Gene ID: 23531), MMP14 (Entrez Gene ID: 4323), MMP7 (Entrez Gene ID: 4316), MOB3A (Entrez Gene ID: 126308), MSC (Entrez Gene ID: 9242), MSRB1 (Entrez Gene ID: 51734), MT1F (Entrez Gene ID: 4494), MTRNR2L8 (Entrez Gene ID: 100463486), MTRNR2L9 (Entrez Gene ID: 100463487), MXD (Entrez Gene ID: 83463), MXD4 (Entrez Gene ID: 10608), MYO10 (Entrez Gene ID: 4651), MYO1D (Entrez Gene ID: 4642), MYO6 (Entrez Gene ID: 4646), MYO6 (Entrez Gene ID: 4646), NAGK (Entrez Gene ID: 55577), NCOA7 (Entrez Gene ID: 135112), NDRG1 (Entrez Gene ID: 10397), NEDD9 (Entrez Gene ID: 4739), NES (Entrez Gene ID: 10763), NEU1 (Entrez Gene ID: 4758), NEURL3 (Entrez Gene ID: 93082), NFKB2 (Entrez Gene ID: 4791), NFKBIA (Entrez Gene ID: 4792), NFKBIE (Entrez Gene ID: 4794), NFKBIZ (Entrez Gene ID: 64332), NGEF (Entrez Gene ID: 25791), NINL (Entrez Gene ID: 22981), NLRP1 (Entrez Gene ID: 22861), NMU (Entrez Gene ID: 10874), NRCAM (Entrez Gene ID: 4897), NRP1 (Entrez Gene ID: 8829), NTF3 (Entrez Gene ID: 4908), OCIAD2 (Entrez Gene ID: 132299), ODF3B (Entrez Gene ID: 440836), OPLAH (Entrez Gene ID: 26873), OPN3 (Entrez Gene ID: 23596), OPTN (Entrez Gene ID: 10133), OR11H6 (Entrez Gene ID: 122748), OR2G2 (Entrez Gene ID: 81470), OR5R1 (Entrez Gene ID: 219479), OSBPL2 (Entrez Gene ID: 9885), OSMR (Entrez Gene ID: 9180), OSR1 (Entrez Gene ID: 130497), OTUD1 (Entrez Gene ID: 220213), P2RY6 (Entrez Gene ID: 5031), PAK6 (Entrez Gene ID: 56924), PANX2 (Entrez Gene ID: 56666), PAPL (Entrez Gene ID: 390928), PARP10 (Entrez Gene ID: 84875), PBX4 (Entrez Gene ID: 80714), PCSK9 (Entrez Gene ID: 255738), PDESA (Entrez Gene ID: 8654), PDK3 (Entrez Gene ID: 5165), PDZK1IP1 (Entrez Gene ID: 10158), PERP (Entrez Gene ID: 64065), PFN2 (Entrez Gene ID: 5217), PGM3 (Entrez Gene ID: 5238), PINK1 (Entrez Gene ID: 65018), PIR (Entrez Gene ID: 8544), PLA2G16 (Entrez Gene ID: 11145), PLA2G7 (Entrez Gene ID: 7941), PLAU (Entrez Gene ID: 5328), PLEKHF1 (Entrez Gene ID: 79156), PLEKHG6 (Entrez Gene ID: 55200), PLGLB1 (Entrez Gene ID: 5343), PMEPA1 (Entrez Gene ID: 56937), PNPLA8 (Entrez Gene ID: 50640), PNRC1 (Entrez Gene ID: 10957), POPDC3 (Entrez Gene ID: 64208), PPAP2B (Entrez Gene ID: 8613), PPAP2C (Entrez Gene ID: 8612), PRELP (Entrez Gene ID: 5549), PRSS8 (Entrez Gene ID: 5652), PSAP (Entrez Gene ID: 5660), PSENEN (Entrez Gene ID: 55851), PSMB9 (Entrez Gene ID: 5698), PSTPIP2 (Entrez Gene ID: 9050), PTGES (Entrez Gene ID: 9536), PTGR1 (Entrez Gene ID: 22949), PTPN6 (Entrez Gene ID: 5777), PTPRB (Entrez Gene ID: 5787), PTPRR (Entrez Gene ID: 5801), PTX4 (Entrez Gene ID: 390667), PXDN (Entrez Gene ID: 7837), RAB11FIP4 (Entrez Gene ID: 84440), RAB17 (Entrez Gene ID: 64284), RAB42 (Entrez Gene ID: 115273), RAI14 (Entrez Gene ID: 26064), RARRES1 (Entrez Gene ID: 5918), RASSF4 (Entrez Gene ID: 83937), RBKS (Entrez Gene ID: 64080), RBP1 (Entrez Gene ID: 5947), RBP1 (Entrez Gene ID: 5947), RBP7 (Entrez Gene ID: 116362), RELB (Entrez Gene ID: 5971), RGCC (Entrez Gene ID: 28984), RGS10 (Entrez Gene ID: 6001), RIBC2 (Entrez Gene ID: 26150), RND1 (Entrez Gene ID: 27289), RNF157 (Entrez Gene ID: 114804), RNF44 (Entrez Gene ID: 22838), RNFS (Entrez Gene ID: 6048), RNF5P1 (Entrez Gene ID: 286140), RUFY2 (Entrez Gene ID: 55680), S100A9 (Entrez Gene ID: 6280), S100P (Entrez Gene ID: 6286), S1PR2 (Entrez Gene ID: 9294), SAA1 (Entrez Gene ID: 6288), SAT1 (Entrez Gene ID: 6303), SCX (Entrez Gene ID: 642658), SDCBP (Entrez Gene ID: 6386), SDCBP2 (Entrez Gene ID: 27111), SEMA3C (Entrez Gene ID: 10512), SEMA4B (Entrez Gene ID: 10509), SERPINA (Entrez Gene ID: 12), SERPINBS (Entrez Gene ID: 5268), SERPINE1 (Entrez Gene ID: 5054), SERTAD4 (Entrez Gene ID: 56256), SGPL1 (Entrez Gene ID: 8879), SH3KBP1 (Entrez Gene ID: 30011), SH3PXD2B (Entrez Gene ID: 285590), SHROOM2 (Entrez Gene ID: 357), SLC11A2 (Entrez Gene ID: 4891), SLC16A2 (Entrez Gene ID: 6567), SLC1A3 (Entrez Gene ID: 6507), SLC35D2 (Entrez Gene ID: 11046), SLC7A7 (Entrez Gene ID: 9056), SLC9A7 (Entrez Gene ID: 84679), SLC9A7P1 (Entrez Gene ID: 121456), SLFNS (Entrez Gene ID: 162394), SLITRK6 (Entrez Gene ID: 84189), SLPI (Entrez Gene ID: 6590), SMO (Entrez Gene ID: 6608), SMOC2 (Entrez Gene ID: 64094), SNN (Entrez Gene ID: 8303), SNORA4 (Entrez Gene ID: 619568), SNORA63 (Entrez Gene ID: 6043), SORCS2 (Entrez Gene ID: 57537), SOX4 (Entrez Gene ID: 6659), SOX9 (Entrez Gene ID: 6662), SPAG4 (Entrez Gene ID: 6676), SPATA17 (Entrez Gene ID: 128153), SPATS2L (Entrez Gene ID: 26010), SPEF1 (Entrez Gene ID: 25876), SPINT1 (Entrez Gene ID: 6692), SPSB3 (Entrez Gene ID: 90864), SPTSSB (Entrez Gene ID: 165679), SQRDL (Entrez Gene ID: 58472), SQSTM1 (Entrez Gene ID: 8878), ST6GAL1 (Entrez Gene ID: 6480), STAT3 (Entrez Gene ID: 6774), STATSA (Entrez Gene ID: 6776), STBD1 (Entrez Gene ID: 8987), STEAP4 (Entrez Gene ID: 79689), SULF2 (Entrez Gene ID: 55959), SULT1E1 (Entrez Gene ID: 6783), SUSD2 (Entrez Gene ID: 56241), SUSD3 (Entrez Gene ID: 203328), SVIL (Entrez Gene ID: 6840), SYTL4 (Entrez Gene ID: 94121), TCEA3 (Entrez Gene ID: 6920), TCTEX1D4 (Entrez Gene ID: 343521), TES (Entrez Gene ID: 26136), 1′EX40 (Entrez Gene ID: 25858), TLR2 (Entrez Gene ID: 7097), TMEM102 (Entrez Gene ID: 284114), TMEM154 (Entrez Gene ID: 201799), TMEM159 (Entrez Gene ID: 57146), TMEM191A (Entrez Gene ID: 84222), TMEM191B (Entrez Gene ID: 728229), TMEM191C (Entrez Gene ID: 645426), TNC (Entrez Gene ID: 3371), TNF (Entrez Gene ID: 7124), TNFAIP2 (Entrez Gene ID: 7127), TNFAIP3 (Entrez Gene ID: 7128), TNFAIP8 (Entrez Gene ID: 25816), TNFAIP8L1 (Entrez Gene ID: 126282), TNFSF10 (Entrez Gene ID: 8743), TNIP1 (Entrez Gene ID: 10318), TNS1 (Entrez Gene ID: 7145), TOR4A (Entrez Gene ID: 54863), TP53TG1(Entrez Gene ID: 11257), TPBG (Entrez Gene ID: 7162), TRIM31 (Entrez Gene ID: 11074), TRIM36 (Entrez Gene ID: 55521), TRIMS (Entrez Gene ID: 85363), TRIM58 (Entrez Gene ID: 25893), TRIM6 (Entrez Gene ID: 117854), TSPAN13(Entrez Gene ID: 27075), TSPAN15 (Entrez Gene ID: 23555), TSPAN8 (Entrez Gene ID: 7103), TTC39A (Entrez Gene ID: 22996), UCA1 (Entrez Gene ID: 652995), UGT1A6 (Entrez Gene ID: 54578), USB1 (Entrez Gene ID: 79650), VAMP8 (Entrez Gene ID: 8673), VAT1 (Entrez Gene ID: 10493), VAV3 (Entrez Gene ID: 10451), VWA1 (Entrez Gene ID: 64856), WIPF1 (Entrez Gene ID: 7456), WISP2 (Entrez Gene ID: 8839), WNT10A (Entrez Gene ID: 80326), WWOX (Entrez Gene ID: 51741), XCL1 (Entrez Gene ID: 6375), XCL2 (Entrez Gene ID: 6846), YPELS (Entrez Gene ID: 51646), ZBTB4 (Entrez Gene ID: 57659), ZC3H12A (Entrez Gene ID: 80149), ZG16 (Entrez Gene ID: 653808), ZNF114 (Entrez Gene ID: 163071), ZNF204P (Entrez Gene ID: 7754), ZNF239 (Entrez Gene ID: 8187), ZNF32 (Entrez Gene ID: 7580), ZNF799 (Entrez Gene ID: 90576), ZNF853 (Entrez Gene ID: 54753), and ZSCAN31 (Entrez Gene ID: 64288).

The resistance modulating agent can promote or upregulate expression of at least one of the following genes, as identified by Entrez Gene IDs according to the NCBI gene database (www.ncbi.nlm.nih.gov/entrez/query.fcgi?db=gene): AASS (Entrez Gene ID: 10157), ABCB1 (Entrez Gene ID: 5243), ABCC4(Entrez Gene ID: 10257), ACACB (Entrez Gene ID: 32), ACAP1 (Entrez Gene ID: 9744), ACOX2 (Entrez Gene ID: 8309), ACSL4 (Entrez Gene ID: 2182), ACTA2 (Entrez Gene ID: 59), ACTG2 (Entrez Gene ID: 72), ACTL8 (Entrez Gene ID: 81569), ADAM19 (Entrez Gene ID: 8728), ADAMTS16 (Entrez Gene ID: 170690), ADAMTSL1 (Entrez Gene ID: 92949), ADARB1 (Entrez Gene ID: 104), ALG1L9P (Entrez Gene ID: 285407), ALPK2 (Entrez Gene ID: 115701), AMOT (Entrez Gene ID: 154796), AMPH (Entrez Gene ID: 273), ANAPC1 (Entrez Gene ID: 64682), ANK1 (Entrez Gene ID: 286), ANKMY1 (Entrez Gene ID: 51281), ANKRD28 (Entrez Gene ID: 23243), ANO2 (Entrez Gene ID: 57101), ANXA6 (Entrez Gene ID: 309), AOC3 (Entrez Gene ID: 8639), APLP1 (Entrez Gene ID: 333), APOOL (Entrez Gene ID: 139322), ARHGAP28 (Entrez Gene ID: 79822), ARHGAP44 (Entrez Gene ID: 9912), ARNT2 (Entrez Gene ID: 9915), ATAD3B (Entrez Gene ID: 83858), ATP6V0A2 (Entrez Gene ID: 23545), BACE2 (Entrez Gene ID: 25825), BAIAP2L2 (Entrez Gene ID: 80115), BCKDHB (Entrez Gene ID: 594), BHLHE41 (Entrez Gene ID: 79365), BMPER (Entrez Gene ID: 168667), BNC1 (Entrez Gene ID: 646), BST2 (Entrez Gene ID: 684), C19orf33 (Entrez Gene ID: 64073), C1orf110 (Entrez Gene ID: 339512), C1orf198 (Entrez Gene ID: 84886), C1QTNF9B-AS1 (Entrez Gene ID: 542767), C21orf91 (Entrez Gene ID: 54149), C2CD2 (Entrez Gene ID: 25966), C7orf31 (Entrez Gene ID: 136895), C9orf64 (Entrez Gene ID: 84267), CALD1 (Entrez Gene ID: 800), CALD1 (Entrez Gene ID: 800), CAMK1D (Entrez Gene ID: 57118), CAMK2D (Entrez Gene ID: 817), CAMK2D (Entrez Gene ID: 817), CAMK2D (Entrez Gene ID: 817), CARD11 (Entrez Gene ID: 84433), CCDC136 (Entrez Gene ID: 64753), CCDC170 (Entrez Gene ID: 80129), CCDC66 (Entrez Gene ID: 285331), CCDC69 (Entrez Gene ID: 26112), CCDC85A (Entrez Gene ID: 114800), CCDC85A (Entrez Gene ID: 114800), CCK (Entrez Gene ID: 885), CCL2 (Entrez Gene ID: 6347), CCL2 (Entrez Gene ID: 6347), CCLS (Entrez Gene ID: 6352), CCND1 (Entrez Gene ID: 595), CD300C (Entrez Gene ID: 10871), CD33 (Entrez Gene ID: 945), CD40 (Entrez Gene ID: 958), CD40 (Entrez Gene ID: 958), CDH18 (Entrez Gene ID: 1016), CDX2 (Entrez Gene ID: 1045), CECRS-AS1 (Entrez Gene ID: 100130717), CESSA (Entrez Gene ID: 221223), CETP (Entrez Gene ID: 1071), CFAP58 (Entrez Gene ID: 159686), CFD (Entrez Gene ID: 1675), CITED1 (Entrez Gene ID: 4435), CKMT1A (Entrez Gene ID: 548596), CKMT1B (Entrez Gene ID: 1159), CLCN4 (Entrez Gene ID: 1183), CLCN6 (Entrez Gene ID: 1185), CLDN25 (Entrez Gene ID: 644672), CLYBL (Entrez Gene ID: 171425), CMTM8 (Entrez Gene ID: 152189), CNN1 (Entrez Gene ID: 1264), CNRIP1 (Entrez Gene ID: 25927), CNRIP1 (Entrez Gene ID: 25927), COL8A1 (Entrez Gene ID: 1295), COPS2 (Entrez Gene ID: 9318), CPA2 (Entrez Gene ID: 1358), CPA3 (Entrez Gene ID: 1359), CPQ (Entrez Gene ID: 10404), CREB3L1 (Entrez Gene ID: 90993), CRLF2 (Entrez Gene ID: 64109), CST6 (Entrez Gene ID: 1474), CTGF (Entrez Gene ID: 1490), CUL4B (Entrez Gene ID: 8450), CYP1A1 (Entrez Gene ID: 1543), CYSTM1 (Entrez Gene ID: 84418), DAB2 (Entrez Gene ID: 1601), DCLK2 (Entrez Gene ID: 166614), DDAH1 (Entrez Gene ID: 23576), DDX3Y (Entrez Gene ID: 8653), DIAPH2 (Entrez Gene ID: 1730), DISP1 (Entrez Gene ID: 84976), DLC1 (Entrez Gene ID: 10395), DNAAF3 (Entrez Gene ID: 352909), DNAJC15 (Entrez Gene ID: 29103), DNAJC22 (Entrez Gene ID: 79962), DOCK11 (Entrez Gene ID: 139818), DPYD (Entrez Gene ID: 1806), DPYD (Entrez Gene ID: 1806), DSC3 (Entrez Gene ID: 1825), DUSP22 (Entrez Gene ID: 56940), DUSP7 (Entrez Gene ID: 1849), DYRK2 (Entrez Gene ID: 8445), EBF3 (Entrez Gene ID: 253738), EDNRB (Entrez Gene ID: 1910), EDNRB (Entrez Gene ID: 1910), EEF1A1 (Entrez Gene ID: 1915), EFNB3 (Entrez Gene ID: 1949), EGFLAM (Entrez Gene ID: 133584), EGR1 (Entrez Gene ID: 1958), EGR3 (Entrez Gene ID: 1960), EID1 (Entrez Gene ID: 23741), ELP6 (Entrez Gene ID: 54859), EMP1 (Entrez Gene ID: 2012), ENG (Entrez Gene ID: 2022), ENOX1 (Entrez Gene ID: 55068), EPB41L3 (Entrez Gene ID: 23136), EPB41L4A (Entrez Gene ID: 64097), EPB41L5 (Entrez Gene ID: 57669), EPN3 (Entrez Gene ID: 55040), EVA1A (Entrez Gene ID: 84141), EXOSC7 (Entrez Gene ID: 23016), EXOSC7 (Entrez Gene ID: 23016), FADS3 (Entrez Gene ID: 3995), FAH (Entrez Gene ID: 2184), FAM110B (Entrez Gene ID: 90362), FAM122B (Entrez Gene ID: 159090), FAM124A (Entrez Gene ID: 220108), FAM129A (Entrez Gene ID: 116496), FAM205BP (Entrez Gene ID: 389715), FAM216A (Entrez Gene ID: 29902), FAM58A (Entrez Gene ID: 92002), FAM78B (Entrez Gene ID: 149297), FBN2 (Entrez Gene ID: 2201), FBXL7(Entrez Gene ID: 23194), FBXO4 (Entrez Gene ID: 26272), FERMT2 (Entrez Gene ID: 10979), FGF2 (Entrez Gene ID: 2247), FGFR3 (Entrez Gene ID: 2261), FHL1 (Entrez Gene ID: 2273), FHL1 (Entrez Gene ID: 2273), FJX1 (Entrez Gene ID: 24147), FMR1(Entrez Gene ID: 2332), FOXA2 (Entrez Gene ID: 3170), FOXP1 (Entrez Gene ID: 27086), FOXP1 (Entrez Gene ID: 27086), FSTL3 (Entrez Gene ID: 10272), FUT4 (Entrez Gene ID: 2526), GAB3 (Entrez Gene ID: 139716), GAL3ST3 (Entrez Gene ID: 89792), GATB (Entrez Gene ID: 5188), GATM (Entrez Gene ID: 2628), GEM (Entrez Gene ID: 2669), GJA1 (Entrez Gene ID: 2697), GLI2 (Entrez Gene ID: 2736), GLIPR1 (Entrez Gene ID: 11010), GLIPR1 (Entrez Gene ID: 11010), GLIPR2 (Entrez Gene ID: 152007), GLRB (Entrez Gene ID: 2743), GMPR (Entrez Gene ID: 2766), GNG11 (Entrez Gene ID: 2791), GPC4 (Entrez Gene ID: 2239), GPD1L (Entrez Gene ID: 23171), GPHN (Entrez Gene ID: 10243), GPR176 (Entrez Gene ID: 11245), HBEGF (Entrez Gene ID: 1839), HIST1H2AC (Entrez Gene ID: 8334), HIST1H2BD (Entrez Gene ID: 3017), HIST1H2BD (Entrez Gene ID: 3017), HIST1H2BO (Entrez Gene ID: 8348), HIST2H4A (Entrez Gene ID: 8370), HIST2H4B (Entrez Gene ID: 554313), HOXB2 (Entrez Gene ID: 3212), HRASLS (Entrez Gene ID: 57110), HRCT1 (Entrez Gene ID: 646962), HTRA1 (Entrez Gene ID: 5654), ID2 (Entrez Gene ID: 3398), IFITM2 (Entrez Gene ID: 10581), IFRD2 (Entrez Gene ID: 7866), IGF2 (Entrez Gene ID: 3481), IGFBP7 (Entrez Gene ID: 3490), IL12A (Entrez Gene ID: 3592), IL17RE (Entrez Gene ID: 132014, INPP5J (Entrez Gene ID: 27124), INS-IGF2 (Entrez Gene ID: 723961), IZUMO3 (Entrez Gene ID: 100129669), KANK1 (Entrez Gene ID: 23189), KANK4 (Entrez Gene ID: 163782), KANK4 (Entrez Gene ID: 163782), KAT2B (Entrez Gene ID: 8850), KCNJ18 (Entrez Gene ID: 100134444), KCNS2 (Entrez Gene ID: 3788), KCTD12 (Entrez Gene ID: 115207), KIAA1109 (Entrez Gene ID: 84162), KLF9 (Entrez Gene ID: 687), KLHDC7A (Entrez Gene ID: 127707), KLHDC8B (Entrez Gene ID: 200942), KLK6 (Entrez Gene ID: 5653), KRT13 (Entrez Gene ID: 3860), LACTB2 (Entrez Gene ID: 51110), LATS2 (Entrez Gene ID: 26524), LCE1E (Entrez Gene ID: 353135), LDB2 (Entrez Gene ID: 9079), LDLRAD3 (Entrez Gene ID: 143458), LGI3 (Entrez Gene ID: 203190), LINC00858 (Entrez Gene ID: 170425), LINC01312 (Entrez Gene ID: 154089), LIX1L (Entrez Gene ID: 128077), LOC100128164 (Entrez Gene ID: 100128164), LOC100506929 (Entrez Gene ID: 100506929), LOC101060179 (Entrez Gene ID: 101060179), LOC151760 (Entrez Gene ID: 151760), LOC653602 (Entrez Gene ID: 653602), LOX (Entrez Gene ID: 4015), LOX (Entrez Gene ID: 4015), LOXL1 (Entrez Gene ID: 4016), LRP12 (Entrez Gene ID: 29967), LRP4 (Entrez Gene ID: 4038), LRRC26 (Entrez Gene ID: 389816), LRRC8C (Entrez Gene ID: 84230), LSM11 (Entrez Gene ID: 134353), LY6D (Entrez Gene ID: 8581), MACROD1 (Entrez Gene ID: 28992), MAGED1 (Entrez Gene ID: 9500), MANF (Entrez Gene ID: 7873), MAP1B (Entrez Gene ID: 4131), MAP2 (Entrez Gene ID: 4133), MAP7D3 (Entrez Gene ID: 79649), MARCH4 (Entrez Gene ID: 57574), MARCH9 (Entrez Gene ID: 92979), MASP1 (Entrez Gene ID: 5648), MATN2 (Entrez Gene ID: 4147), MCTS1 (Entrez Gene ID: 28985), MEF2C (Entrez Gene ID: 4208), METTL24 (Entrez Gene ID: 728464), MKRN3 (Entrez Gene ID: 7681), MMGT1 (Entrez Gene ID: 93380), MMP1 (Entrez Gene ID: 4312), MN1 (Entrez Gene ID: 4330), MPP7 (Entrez Gene ID: 143098), MRGPRX3 (Entrez Gene ID: 117195), MSRB3 (Entrez Gene ID: 253827), MSRB3 (Entrez Gene ID: 253827), MTFMT (Entrez Gene ID: 123263), MYLS (Entrez Gene ID: 4636), MYOM2 (Entrez Gene ID: 9172), MZT1 (Entrez Gene ID: 440145), NANOS1 (Entrez Gene ID: 340719), NAV1 (Entrez Gene ID: 89796), NCR3LG1 (Entrez Gene ID: 374383), NDUFA4 (Entrez Gene ID: 4697), NDUFAF4 (Entrez Gene ID: 29078), NDUFAF4P1 (Entrez Gene ID: 100306975), NDUFC1 (Entrez Gene ID: 4717), NEK4 (Entrez Gene ID: 6787), NEO1 (Entrez Gene ID: 4756), NFATC2IP (Entrez Gene ID: 84901), NFIX (Entrez Gene ID: 4784), NGF (Entrez Gene ID: 4803), NLK (Entrez Gene ID: 51701), NNMT (Entrez Gene ID: 4837), NR2F1 (Entrez Gene ID: 7025), NREP (Entrez Gene ID: 9315), NRG1 (Entrez Gene ID: 3084), NRG4 (Entrez Gene ID: 145957), NRXN3 (Entrez Gene ID: 9369), NUP210 (Entrez Gene ID: 23225), OAS1 (Entrez Gene ID: 4938), OAZ3 (Entrez Gene ID: 51686), OR2B6 (Entrez Gene ID: 26212), OR4C46 (Entrez Gene ID: 119749), OR6N2 (Entrez Gene ID: 81442), OR7G2 (Entrez Gene ID: 390882), OSGEP (Entrez Gene ID: 55644), P3H3 (Entrez Gene ID: 10536), PAPPA2 (Entrez Gene ID: 60676), PARP8 (Entrez Gene ID: 79668), PCDH18 (Entrez Gene ID: 54510), PCLO (Entrez Gene ID: 27445), PCOLCE2 (Entrez Gene ID: 26577), PDE4B (Entrez Gene ID: 5142), PDGFRB (Entrez Gene ID: 5159), PDZD4 (Entrez Gene ID: 57595), PEAK1 (Entrez Gene ID: 79834), PFKM (Entrez Gene ID: 5213), PHLDA1 (Entrez Gene ID: 22822), PITPNB (Entrez Gene ID: 23760), PITPNB (Entrez Gene ID: 23760), PITX2 (Entrez Gene ID: 5308), PKDCC (Entrez Gene ID: 91461), PLA2G4F (Entrez Gene ID: 255189), PLBD1 (Entrez Gene ID: 79887), PLEKHG4 (Entrez Gene ID: 25894), PLEKHGS (Entrez Gene ID: 57449), PMP22 (Entrez Gene ID: 5376), PMVK (Entrez Gene ID: 10654), POLR3G (Entrez Gene ID: 10622), PPAPDC1A (Entrez Gene ID: 196051), PPARGC1B (Entrez Gene ID: 133522), PRAME (Entrez Gene ID: 23532), PRDM16 (Entrez Gene ID: 63976), PRICKLE2 (Entrez Gene ID: 166336), PRKCA (Entrez Gene ID: 5578), PRSS12 (Entrez Gene ID: 8492), PRUNE2 (Entrez Gene ID: 158471), PTPRM (Entrez Gene ID: 5797), PURA (Entrez Gene ID: 5813), PVRL3 (Entrez Gene ID: 25945), QPCT (Entrez Gene ID: 25797), RARB (Entrez Gene ID: 5915), RBMS3 (Entrez Gene ID: 27303), RCBTB2 (Entrez Gene ID: 1102), RCHY1 (Entrez Gene ID: 25898), RFTN1 (Entrez Gene ID: 23180), RGS2 (Entrez Gene ID: 5997), RGS7 (Entrez Gene ID: 6000), RIMKLB (Entrez Gene ID: 57494), RIMS2 (Entrez Gene ID: 9699), RIMS3 (Entrez Gene ID: 9783), RIOK2 (Entrez Gene ID: 55781), RNASEH2B (Entrez Gene ID: 79621), RNF150 (Entrez Gene ID: 57484), RNF182 (Entrez Gene ID: 221687), RPRD1A (Entrez Gene ID: 55197), RPRML (Entrez Gene ID: 388394), RPS19 (Entrez Gene ID: 6223), RPS26 (Entrez Gene ID: 6231), RRP15 (Entrez Gene ID: 51018), RSL1D1 (Entrez Gene ID: 26156), RUNDC3B (Entrez Gene ID: 154661), RYBP (Entrez Gene ID: 23429), S1PR5(Entrez Gene ID: 53637), SACM1L (Entrez Gene ID: 22908), SAMD11 (Entrez Gene ID: 148398), SCAF1 (Entrez Gene ID: 58506), SCARA3 (Entrez Gene ID: 51435), SCG2 (Entrez Gene ID: 7857), SDC2 (Entrez Gene ID: 6383), SDHD (Entrez Gene ID: 6392), SDPR (Entrez Gene ID: 8436), SEMA3F (Entrez Gene ID: 6405), SEPT4 (Entrez Gene ID: 5414), SERPINH1 (Entrez Gene ID: 871), SFMBT2 (Entrez Gene ID: 57713), SH3BGR (Entrez Gene ID: 6450), SHROOM4 (Entrez Gene ID: 57477), SIMC1 (Entrez Gene ID: 375484), SLC22A31 (Entrez Gene ID: 146429), SLC25A15 (Entrez Gene ID: 10166), SLC25A3P1 (Entrez Gene ID: 163742), SLC25A43 (Entrez Gene ID: 203427), SLC26A10 (Entrez Gene ID: 65012), SLC2A5 (Entrez Gene ID: 6518), SLC39A14 (Entrez Gene ID: 23516), SLC4A8 (Entrez Gene ID: 9498), SLC7A2 (Entrez Gene ID: 6542), SLFN11 (Entrez Gene ID: 91607), SMARCD3 (Entrez Gene ID: 6604), SMCO4 (Entrez Gene ID: 56935), SMIM23 (Entrez Gene ID: 644994), SNCA (Entrez Gene ID: 6622), SOBP (Entrez Gene ID: 55084), SOX18 (Entrez Gene ID: 54345), SOX6 (Entrez Gene ID: 55553), SP140 (Entrez Gene ID: 11262), SRSF8 (Entrez Gene ID: 10929), SS18L2 (Entrez Gene ID: 51188), SSBP2 (Entrez Gene ID: 23635), SSSCA1 (Entrez Gene ID: 10534), STK19 (Entrez Gene ID: 8859), STK39 (Entrez Gene ID: 27347), STXBP1 (Entrez Gene ID: 6812), STXBP6 (Entrez Gene ID: 29091), SULF1 (Entrez Gene ID: 23213), SYNC (Entrez Gene ID: 81493), TAF1 (Entrez Gene ID: 6872), TAF9B (Entrez Gene ID: 51616), TAGLN (Entrez Gene ID: 6876), TCEAL8 (Entrez Gene ID: 90843), TCF4 (Entrez Gene ID: 6925), TENM2 (Entrez Gene ID: 57451), IEX264 (Entrez Gene ID: 51368), TGFB1I1 (Entrez Gene ID: 7041), TGFBR3 (Entrez Gene ID: 7049), THOC7 (Entrez Gene ID: 80145), TIMM8A (Entrez Gene ID: 1678), TIMP3 (Entrez Gene ID: 7078), TLE4 (Entrez Gene ID: 7091), TLR4 (Entrez Gene ID: 7099), TM2D1 (Entrez Gene ID: 83941), TMEM158 (Entrez Gene ID: 25907), TMEM201 (Entrez Gene ID: 199953), TMPRSS11F (Entrez Gene ID: 389208), TMPRSS4 (Entrez Gene ID: 56649), TNNT1 (Entrez Gene ID: 7138), TPM1 (Entrez Gene ID: 7168), TSPAN2 (Entrez Gene ID: 10100), TSPAN3 (Entrez Gene ID: 10099), TUBA1A (Entrez Gene ID: 7846), TXNRD2 (Entrez Gene ID: 10587), UBE2D2 (Entrez Gene ID: 7322), UBE2Q1 (Entrez Gene ID: 55585), UNC13B (Entrez Gene ID: 10497), UTP20 (Entrez Gene ID: 27340), VAMP? (Entrez Gene ID: 6845), VGLL1 (Entrez Gene ID: 51442), VGLL3 (Entrez Gene ID: 389136), VIM (Entrez Gene ID: 7431), VIT (Entrez Gene ID: 5212), VMA21 (Entrez Gene ID: 203547), VPS36 (Entrez Gene ID: 51028), VPS39 (Entrez Gene ID: 23339), VSTM2A (Entrez Gene ID: 222008), WASF3 (Entrez Gene ID: 10810), WNT10B (Entrez Gene ID: 7480), WNTSA (Entrez Gene ID: 7474), XKR7 (Entrez Gene ID: 343702), ZDHHC3 (Entrez Gene ID: 51304), ZEB1 (Entrez Gene ID: 6935), ZFHX4 (Entrez Gene ID: 79776), ZFY (Entrez Gene ID: 7544), ZIC4 (Entrez Gene ID: 84107), ZNF280C (Entrez Gene ID: 55609), ZNF330 (Entrez Gene ID: 27309), ZNF503 (Entrez Gene ID: 84858), ZNF625 (Entrez Gene ID: 90589), and ZNF625-ZNF20 (Entrez Gene ID: 100529855).

The ZVI NPs and the resistance modulating agent(s) can be administered via parenteral, inhalation, local, rectal, nasal, sublingual, or vaginal delivery, or an implanted reservoir. Herein, the term “parenteral delivery” includes subcutaneous, intradermic, intravenous, intra-articular, intra-arterial, synovial, intrapleural, intrathecal, local, and intracranial injections. The ZVI NPs and the resistance modulating agent(s) can be administered separately or in a fixed-dose combination of the two in a single-dosage formulation.

Another aspect of the present invention provides a method for improving efficacy of a cancer treatment. In an embodiment, the method includes the step of: administering an effective amount of at least one of the aforementioned resistance modulating agents to a subject receiving a treatment using the aforementioned ZVI NPs.

Yet another aspect of the present invention provides a method for treating cancer. In an embodiment, the method includes the steps of: obtaining a first transcriptome profile of cancer cells of a subject; providing the cancer cells a prophylactically effective amount of the aforementioned ZVI NPs; obtaining a second transcriptome profile of the cancer cells; and determining susceptibility of the cancer cells to the ZVI NPs according to the difference between the first and second transcriptome profiles; and treating the subject according to the determination result.

The prophylactically effective amount of ZVI NPs is sufficient to induce resistance of the treated cells against the ZVI NPs. The first and second transcriptome profiles may be obtained by RNA extraction followed by microarray analysis. The genes analyzed by the microarray analysis may include, but are not limited to, GSR, AKR1C1, AKR1C3, AKR1B1, AKR1B10, KYNU, ACSL4, ZEB1, NNMT and other upregulated and downregulated genes as listed above. In an embodiment, the difference between the first and second transcriptome profiles may be defined by log 2 (fold change) values and/or log 2 ratios.

In the embodiment, if the cancer cells are determined to be susceptible to the ZVI NPs, the subject is treated by a therapeutically effective amount of ZVI NPs. On the contrary, if the cancer cells are determined to be refractory to the ZVI NPs, the subject is treated by a therapeutically effective amount of ZVI NPs and a therapeutically effective amount of at least one resistance modulating agent. In other words, the method predicts efficacy of ZVI NP treatments on the subject by analyzing the transcriptome profiles of the subject before and after exposure to ZVI NPs, thus offering a reliable reference to treating the subject.

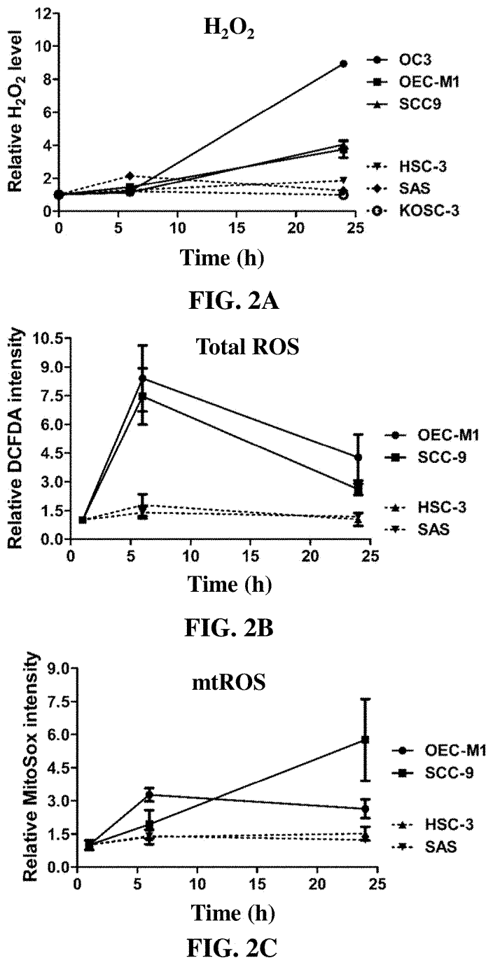

Referring now to FIGS. 2A to 2C. ZVI NP-induced selective cytotoxicity is demonstrated to be associated with production of reactive oxygen species (ROS). Specifically, the amounts of hydrogen peroxide (H₂O₂) generated in ZVI sensitive and refractory cancer cells after treatment with carboxy methyl cellulose (CMC) stabilized ZVI NPs (hereafter referred to as ZVI@CMC NPs) was assessed by photoluminescence detection assay using derivatized luciferin substrates, and as shown in FIG. 2A, the results revealed ZVI@CMC NP treatment could induce intracellular H₂O₂ production in ZVI sensitive cell lines (eg. OC3, OEC-M1 and SCC-9), but not in ZVI refractory cell lines (eg. HSC-3, KOSC-3 and SAS).

Further, ZVI NP-induced intracellular ROS production is shown to elevate only in ZVI sensitive cells. As shown in FIG. 2B, flow cytometry analysis of ZVI sensitive and refractory cells treated with ZVI@CMC NPs for 6 h revealed a significant increase in production of total ROS in sensitive cell lines OEC-M1 and SCC-9. Specifically, the levels of total ROS levels surged for about seven folds after 6 h of treatment, followed by a gradual decline to about 3 folds of the original level in the next 24 h. In contrast, the levels of total ROS in ZVI refractory cell lines HSC-3 and SAS were not affected by the ZVI@CMC NP treatment and maintained at baseline level throughout the treatment.

Similarly, as shown in FIG. 2C, production of mitochondrial ROS (mtROS) is significant only in treated ZVI sensitive cells. The results also suggest that ZVI induced ROS production in the mitochondria or cytoplasm of ZVI sensitive cells is cell line dependent. However, all ZVI refractory cell lines were consistently unaffected by the oxidative challenge caused by ZVI@CMC NPs and were able to maintain the levels of both mtROS and total ROS to the baseline level.

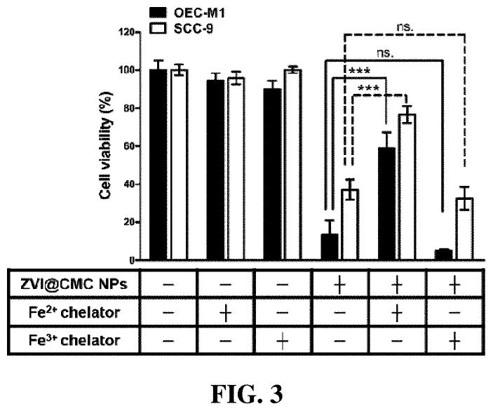

Referring now to FIG. 3. The ZVI NP-induced selective cytotoxicity is demonstrated to be associated with the Fenton reaction. As shown in FIG. 3, cytotoxicity of carboxy methyl cellulose (CMC) stabilized ZVI NPs (hereafter referred to as ZVI@CMC NPs) was attenuated when treating ZVI sensitive cells OEC-M1 and SCC-9 with ZVI@CMC NPs in combination with a ferrous iron (Fe²⁺) chelator (eg. 1,10-phenanthroline). In contrast, when combining ZVI@CMC NPs with a ferric iron (Fe³⁺) chelator (eg. desferoxamine, DFO), cell viability of the treated cells was not restored. The results suggest that Fe(II) ions, rather than Fe(III) ions, played critical roles in generating ZVI@CMC NP induced cytotoxicity. Together with the selective production of H₂O₂ and ROS as shown in FIGS. 3A to 3C, it is evident that ZVI NPs cause selective cytotoxicity by inducing excessive Fenton reaction and ROS production in ZVI sensitive cells.

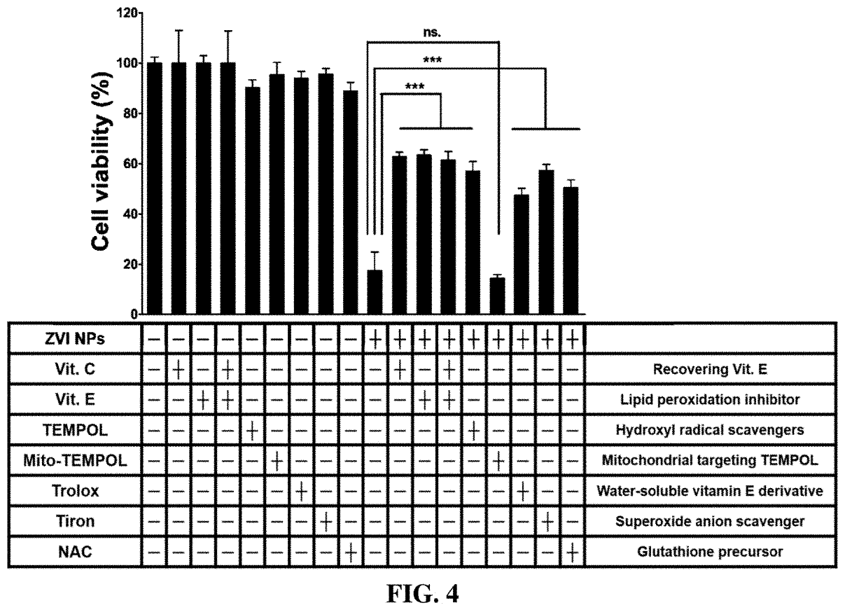

Referring now to FIG. 4. The production of intracellular ROS is demonstrated to be directly responsible for selective cytotoxicity of ZVI NPs. As shown in FIG. 4, when treating ZVI sensitive OEC-M1 cells with ZVI@CMC NPs together with a variety of ROS scavengers (eg. vitamins C and E, 4-hydroxy-2,2,6,6-tetramethylpiperidin-1-oxyl (TEMPOL), 6-Hydroxy-2,5,7,8-tetramethylchroman-2-carboxylic acid (Trolox), and disodium 4,5-dihydroxy-1,3-benzenedisulfonate (Tiron) to deprive common ROS such as hydroxyl radicals (OH.), superoxides (.O₂⁻) and fatty acid radicals in the cells, cell viability of the OEC-M1 cells recovered for about 50% as compared with that when treated with ZVI@CMC NPs alone. The addition of N-acetylcysteine (NAC), which acted as a glutathione (GSH) precursor to supply GSH to the treated OEC-M1 cells, also showed to reduce cytotoxicity of ZVI@CMC NPs to the ZVI sensitive cells. Interestingly, cotreatment of mitochondria targeting antioxidant mito-IEMPOL with ZVI@CMC NPs did not shown to alter ZVI induced cytotoxicity, suggesting that cytosolic ROS rather than mitochondrial ROS played a dominant role in the initiation of ZVI@CMC NP induced cytotoxicity.

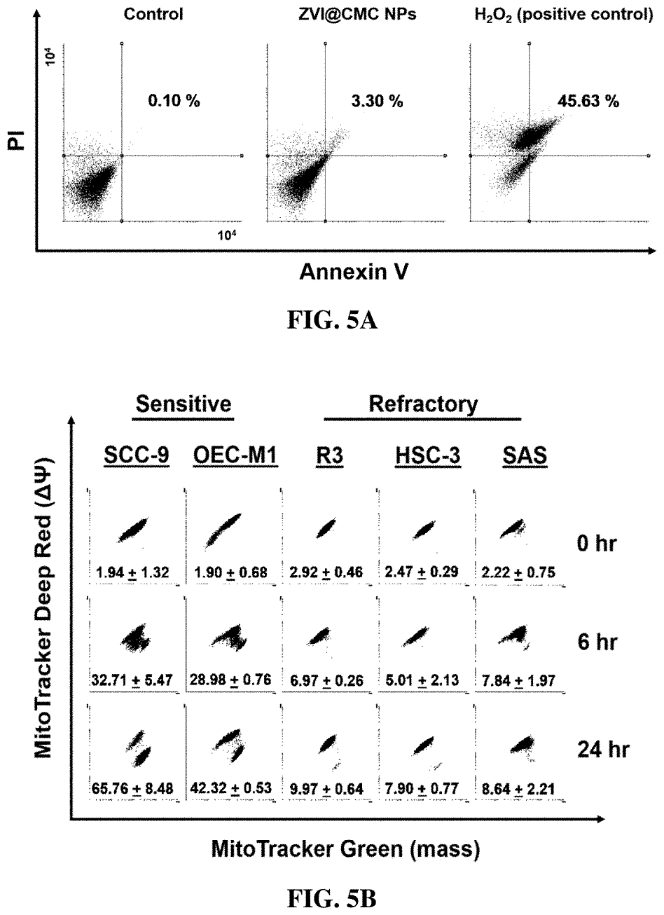

Referring now to FIGS. 5A and 5B. The ZVI NP induced cell death is demonstrated to be non-apoptotic and associated with mitochondria dysfunction. While ROS stress has been known to play a critical role in apoptosis, flow cytometry analysis, as shown in FIG. 5A, revealed that when treated with ZVI@CMC NPs, ZVI sensitive OEC-M1 cells did not exhibit a phosphatidylserine (PS) externalization that is characteristic of an apoptosis. However, loss of mitochondrial membrane potential (ΔΨ) was observed in ZVI sensitive cells. As shown in FIG. 5B, flow cytometry analysis using mitochondria targeting dyes revealed that time-dependent mitochondrial ΔΨ loss was statistically significant only in ZVI sensitive SCC-9 and OEC-M1 cell lines, but not in ZVI refractory HSC-3 and SAS cell lines.

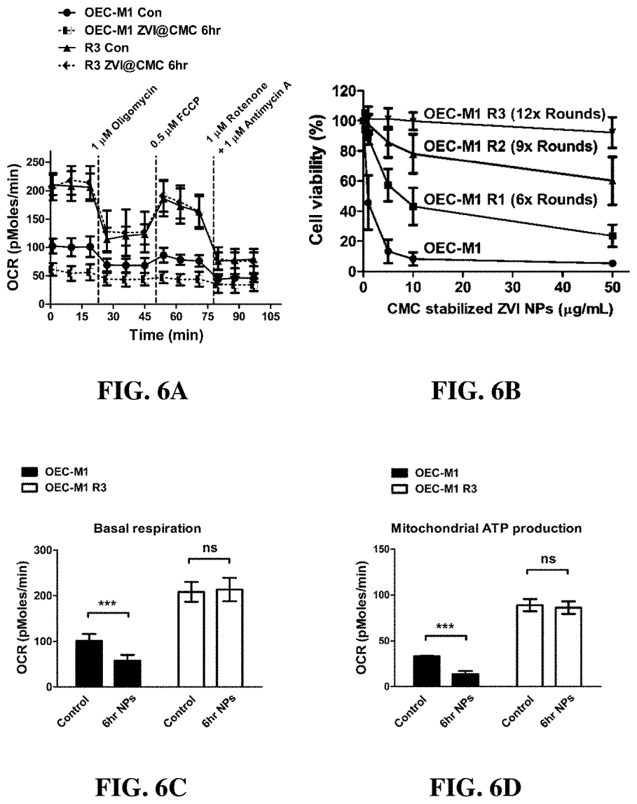

Referring now to FIGS. 6A, 6B, 6C and 6D. To further analyze the ZVI NP induced changes in mitochondrial function, a sensitive and refractory cell pair having the same genomic background were generated, as shown in FIG. 6A, by treating ZVI sensitive OEC-M1 cells with ZVI@CMC NPs at a series of concentrations and collecting the treated cells at different treatment rounds to obtain ZVI refractory OEC-M1 R1, R2 and R3 cell clones, with the OEC-M R3 clone harboring the highest resistance to ZVI NP induced cytotoxicity among the three and the ability to endure ZVI challenge, as shown in FIG. 5B. Furthermore, as shown in FIGS. 6B, 6C and 6D, extracellular flux analysis of the oxygen consumption rate (OCR) and ATP production after oligomycin treatment revealed that the OEC-M1 R3 clone have a significantly higher baseline levels of aerobic respiration and mitochondrial oxidative phosphorylation, as well as increased ATP production, than the ZVI sensitive OEC-M1 cells. The results suggest that ZVI refractory cells escape cell death by scavenging ROS and enhancing mitochondrial pro-survival signals.

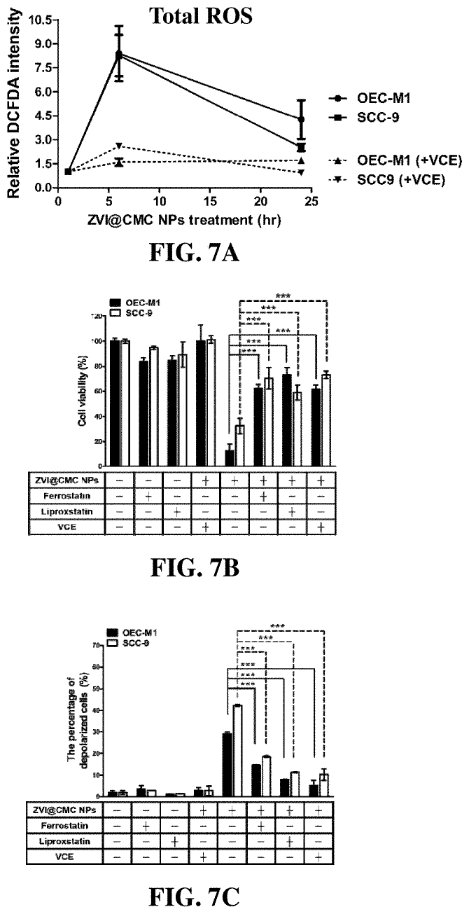

Referring now to FIGS. 7A-7C. The ZVI NP-induced selective cytotoxicity is demonstrated to be conferred by mitochondrial hyperoxidation and induction of ferroptosis in ZVI sensitive cells. As shown in FIG. 7A, cotreatment with lipid peroxidation inhibitors vitamins C and E (denoted as VCE) attenuated ZVI@CMC induced cytotoxicity to and significantly reduced intracellular ROS in ZVI sensitive OEC-M1 and SCC9 cells. Meanwhile, the addition of VCE and ferroptosis inhibitors ferrostatin and liproxstatin was shown to recover the cell viability of ZVI@CMC NP treated OEC-M1 and SCC-9 cells for nearly 50%, as shown in FIG. 7B, and reduce the percentage of depolarized cells caused by ZVI@CMC NP induced mitochondrial hyperoxidation, as shown in FIG. 7C.

Furthermore, fluorescence microscopic analysis (not shown in figures) revealed that ZVI@CMC NP caused accumulation of lipid peroxidation co-localized with mitochondria in OEC-M1 cells, indicating the occurrence of mitochondrial hyperoxidation and thus mitochondrial dysfunction in ZVI sensitive cells. The results also demonstrated that ferroptotic lipid peroxides are generated by mitochondria in ZVI sensitive cells upon ZVI@CMC NP treatment.

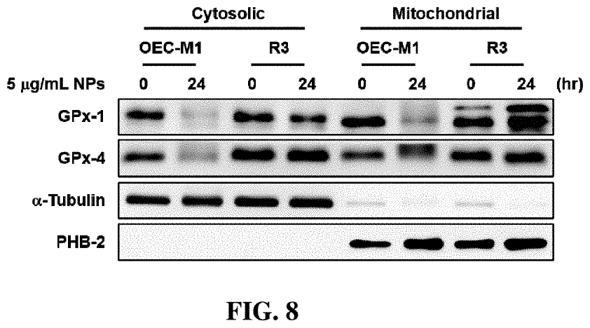

Referring now to FIG. 8. Western blot analysis as shown in FIG. 8 revealed that treatment with ZVI@CMC NPs resulted in reduced levels of cytosolic glutathione peroxidases GPx-1 and GPx-4 in ZVI sensitive OEC-M1 cells; meanwhile, expression of GPx-1 in the mitochondria was also suppressed. On the contrary, in ZVI refractory OEC-M1 R3 cells, the levels of GPx-1 and GPx-4 in the cytosol or mitochondria were unaltered after treatment with ZVI@CMC NPs.

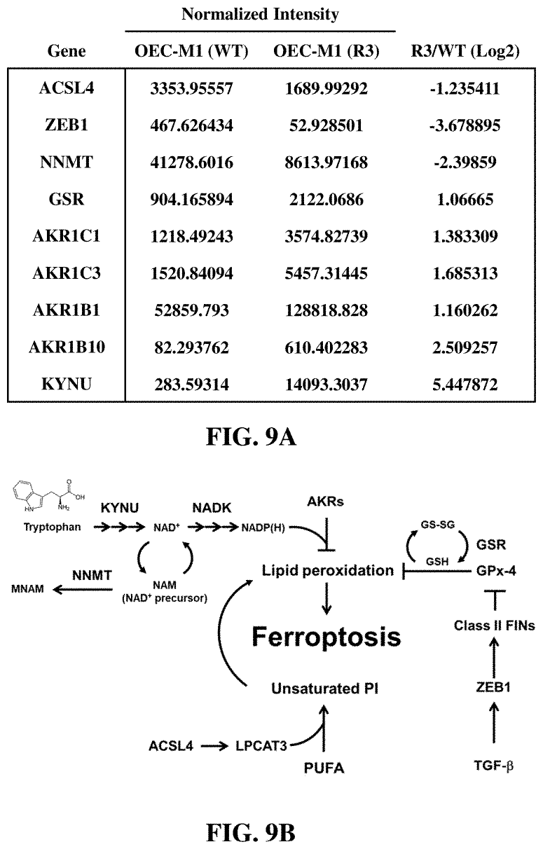

Referring now to FIG. 9A. Ferroptosis resistance associated genes is identified by transcriptome analysis. As shown in FIG. 9A, analysis of the transcriptome profile of the ZVI sensitive/refractory OEC-M1 and OEC-M1 R3 cell pair after treatment with ZVI@CMC NPs revealed ten genes that are most relevant to ferroptosis resistance, including acyl-CoA synthetase long chain family member 4 (ACSL4), zinc finger E-box-binding homeobox 1 (ZEB1), nicotinamide n-methyltransferase (NNMT), glutathione-disulfide reductase (GSR), aldo-keto reductase 1 (AKR1), and kynureninase (KYNU) genes. Among the identified genes, GSR, AKR1C1, AKR1C3, AKR1B1, AKR1B10, and KYNU are upregulated and ACSL4, ZEB1 and NNMT are downregulated in the ZVI refractory OEC-M1 R3 clone.

Specifically, GSR is known to recycle glutathione (GSH) from GSH disulfide (GS-SG) and is a cofactor for all members of the glutathione peroxidase (GPx) family. As supported by the results shown in FIG. 8, GPx-1 is overexpressed in the cytosol of the OEC-M1 R3 clone under oxidative stress caused by ZVI NP challenge to prevent ferroptosis. AKRs are known to participate in the detoxification of oxidized lipid derivatives, and as illustrated in FIG. 9B. KYNU has been shown to mediate NAD⁺ production, thus promoting NADP(H) production and AKR activity. Meanwhile, NNMT is known to be responsible for NAD⁺ depletion by converting nicotinamide (NAM) to N¹-methylnicotinamide (MNAM), as illustrated in FIG. 9B. Therefore, ferroptosis resistance in ZVI refractory cells is demonstrated to be a result of the transcriptional changes in GSR, AKR1C1, AKR1C3, AKR1B1, AKR1B10, KYNU and NNMT genes that promotes NAD⁺ biosynthesis and retention and thus sustains a NADPH level that is sufficient for AKRs to prevent lipid peroxidation caused by ZVI challenge.

Further, two other downregulated genes as shown in FIG. 9A are also associated with development of ferroptosis resistance. ACSL4 is a ferroptosis inducer that participates in the activation of polyunsaturated fatty acids (PUFA) hydroperoxides. ZEB1 is an epithelial-mesenchymal transition (EMT) regulator and lipogenic factor, and downregulation of ZEB1 has been shown to abolish efficacy of GPx-4 inhibitors. Altogether, the results demonstrate the critical role of ferroptosis in development of ZVI resistance in cancer cells.

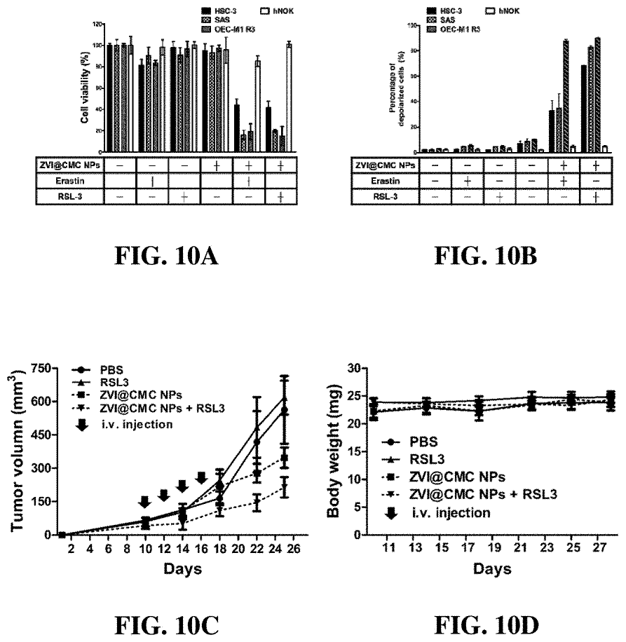

Referring now to FIGS. 10A, 10B, 10C and 10D. Modulating ferroptosis is demonstrated to alter ZVI susceptibility of cancer cells and selectively enhance cytotoxicity of ZVI NPs. As shown in FIG. 10A, when treating ZVI refractory HSC-3, SAS and OEC-M1 R3 cells with ZVI@CMC NPs in combination with Class I ferroptosis inducer (FIN) erastin or Class II FIN RSL-3, cell viability of the treated cells was significantly reduced as compared with those treated with ZVI@CMC NPs alone. Remarkably, cell viability of human normal oral keratinocytes (hNOK) was unaffected by any of the treatments. Meanwhile, when treating the ZVI refractory cells with ZVI@CMC NPs and either of the FINs, the percentage of depolarized cells dramatically increased, indicating significant loss in mitochondrial membrane potential in the treated cells.

The synergistic effect of FINs with ZVI NPs is also demonstrated in vivo. As shown in FIGS. 10B, 10C and 10D, when injecting an xenograft mice model that bears the ZVI refractory SAS cells with Ras selective lethal 3 (RSL-3), a Class II FIN, together with ZVI@CMC NPs, tumor growth of the mice significantly delayed as compared to those treated with ZVI@CMC NPs or RSL-3 alone. Remarkably, body weight of the mice was unaffected by any of the treatments; no other adverse side effect was detected. The results demonstrate that ZVI resistance of cancer cells can be effectively attenuated or overcome by cotreatment with FINs, both in vitro and in vivo.

In the series of experimental demonstrations provided above, the ZVI NPs were prepared as follows: dissolving 0.2 M of iron (II) sulfate in a sodium citrate solution and slowly adding NaBH4 to reduce ferrous ions (Fe²⁺); stirring the ZVI solution at room temperature for 15 min until the solution turns black. To coat the ZVI NPs with gold to prepare ZVI@Au NPs, the ZVI solution was added 0.05 M of HAuCl₄ and stirred at room temperature for 5 min under argon (Ar). To prepare carboxymethyl cellulose (CMC) stabilized ZVI NPs (ZVI@CMC NPs), 0.5 g/L of elemental iron was dissolved in an aqueous solution containing 0.2% of CMC, followed by addition of NaBH₄ (BH⁻/Fe²⁺=4.0) to reduce the Fe′ ions to ZVI. The various nanoparticles are washed with ethanol, collected with a magnet and stored in oxygen-free ethanol.

The OSCC cell lines and human normal oral keratinocytes (hNOK) used in the experimental demonstrations were cultured as follows. Primary hNOK cells were maintained in the Keratinocyte Growth Medium (KGM). The OC2, KOSC-3 and OEC-M1 OSCC cell lines were cultured in the RPMI 1640 medium. The SCC9, HSC-3 and SAS OSCC cell lines were maintained in the DMEM/F-12 medium. The OC3 oral cancer cells were cultured in a mixed medium (DMEM: KGM=2:1). The RPMI 1640, DMEM/F-12 and DMEM were each supplemented with 10% of fetal bovine serum (FBS), 10 μg/mL of streptomycin, 10 U/mL of penicillin and 0.25 μg/mL of amphotericin B. All of the cells were incubated at 37° C. in a humidified atmosphere containing 5% of CO₂ gas.

The cell survival assays in the experimental demonstrations were conducted as follows. Cytotoxicity of the ZVI NPs was evaluated by the MTT assay using 3-(4,5-dimethylthiazol-2-yl)-2,5-diphenyltetrazolium bromide. Log-phase OSCC cells were seeded at a density of 5000 cells per well in a 96-well culture plate and incubated for 16 h. The attached cells were then incubated with a series of concentrations of ZVI NPs for 48 h. Subsequently, culture medium of the cells were replaced by fresh complete medium contained 0.5 mg/mL of MTT and the cells were incubated at 37° C. for 1 h. After removing the medium, the resulting MTT crystals were dissolved in DMSO and measured for their optical absorbance at 550 nm to determine viability of the cells.

Additionally, various cell survival assays were conducted by incubating the cells with 5 μg/mL of ZVI@CMC NPs in combination with an iron chelator (eg. 1 μM of 1, 10-phenanthroline or 1 μM of desferoxamine (DFO)), a ROS scavenger (eg. 100 μM of vitamin C, 100 μM of vitamin E, 100 μM of TEMPOL, 5 μM of mito-lEMPOL, 100 μM Trolox, 100 μM Tiron, or 500 μM NAC), a ferroptosis inhibitor (eg. 5 μM of liproxstatin and 50 μM of ferrostatin) or a ferroptosis inducer (eg. 0.05 μM of RSL-3 or 1 μM of erastin) for 48 h.

In the experimental demonstrations, analyses of the productions of intracellular H₂O₂, total ROS, and mitochondrial ROS were conducted as follows. For measuring H₂O₂, the cells were first seeded on a 96 well plate and incubated for 16 h; thereafter, the cells were treated with 5 μg/mL of ZVI@CMC NPs and 25 μM of ROS-Glo™, a commercial H₂O₂ substrate for a series of duration. Finally, ROS-Glo™ Detection Solution containing a recombinant luciferase and D-Cysteine was added to the cells to obtain luminescent signals that correspond to the concentration of H₂O₂. For measuring ROS, the cells were treated with 5 μg/mL of ZVI@CMC NPs for 6 h and 24 h, followed by incubation with 5 μM of 2′,T-dichlorodihydrofluorescein diacetate (H2DCFDA) for 30 min. The resulting fluorescent 2′7′-dichlorofluorescin (DCF) signals, which is indicative of the production of total ROS in the cells, were measured under an excitation wavelength of 488 nm and an emission wavelength of 515-527 nm and analyzed by a BD Canto II Flow Cytometer. Likewise, the cells treated with ZVI@CMC NPs were incubated with 5 μM of MitoSox, a red dye solution indicative of mitochondrial ROS (mtROS), for 30 min and analyzed for red fluorescent signals under an excitation wavelength of 510 nm and an emission wavelength of 580 nm by a BD Canto II Flow Cytometer.

Cell apoptosis in the experimental demonstrations was analyzed by using annexin-V/PI double staining. Briefly, OEC-M1 cells were untreated (as control) or treated with 100 μM of H₂O₂ (as positive control) or 5 μg/mL of ZVI@CMC NPs for 24 h. Subsequently, the cells were incubated with 10 μg/mL of annexin-V fluorescein isothiocyanate (FITC) and 5 μg/mL of PI in an annexin-V binding buffer for 15 min at room temperature in the dark, and analyzed by a BD Canto II Flow Cytometer.

Depolarization of the ZVI NP treated cells was assessed by double staining with 50 nM of MitoTracker Green, a commercial dye for indicating total mitochondrial mass, and 100 nM of MitoTracker Deep Red, another commercial dye for indicating mitochondria membrane potential (ΔΨ), at 37° C. for 30 min, followed by analysis of red fluorescent signals under an excitation wavelength of 644 nm and an emission wavelength of 665 nm and green fluorescent signals under an excitation wavelength of 490 nm and an emission wavelength of 516 nm by a BD Canto II Flow Cytometer.

Mitochondrial functions of the ZVI NP treated cells were assessed as follows. OEC-M1 and R3 cells were seeded at a density of 25,000 cells/well overnight, and exposed to 5 μg/mL of ZVI@CMC NPs for 6 h. Oxygen consumption rate (OCR) of the cells were analyzed by an XF24 Extracellular Flux Analyzer. Additionally, oligomycin (1 μM), carbonyl cyanide-4-(trifluoromethoxy) phenylhydrazone (FCCP, 0.5 μM) and rotenone (1 μM)+antimycin A (1 μM) were sequentially added into the cells to obtain a dynamic OCR profile for determining basal respiration, ATP production, and respiratory capacity of mitochondria in the cells.

Mitochondrial hyperoxidation and morphological change of the cells were visualized as follows. OEC-M1 cells were first seeded in a 35 mm imaging culture dish for adhesion overnight. For staining of lipid peroxidation, the cells were prestained with 10 μM of LiperFluo for 30 min and treated with 5 μg/mL of ZVI@CMC NPs for 6 h. Thereafter, mitochondria of the cells were stained with 100 nM of MitoTracker Deep Red for 1 h and observed under a DeltaVision Elite microscope equipped with a 100×1.4 NA oil optic, TruLight illumination system, InsightSSI illumination units, Evolve EMCCD and UltimateFocus module. Deconvolution of the images was conducted by a SoftWoRx software.

Western blot analysis of GPx-1 and GPx-4 expressions was performed as follows. OEC-M1 and R3 cells were seeded in 10 cm culture dish for adhesion overnight. The cells were then exposed to 5 μg/mL of ZVI@CMC NPs for 6 h and 24 h. Crude mitochondrial fraction of the treated cells was prepared by differential centrifugation. The cells were harvested and resuspended in SHE buffer (containing protease inhibitor, 0.25 M of sucrose, 0.5 mM of EGTA, 0.5 mM of EDTA, and 3 mM of HEPES in PBS; pH 7.2). A pre-cooled glass dounce tissue grinder was used to homogenize the cells without damaging the mitochondria. The cell lysate was centrifuged at 800 g for 10 min at 4° C. to remove cell debris that contains nucleus and plasma membrane. The supernatant was pooled and centrifuged at 10,000 g for another 30 min at 4° C., resulting in a supernatant that contains the cytosolic fraction and a pellet that contains the mitochondria fraction. The cytosolic fraction was analyzed by western blotting using anti-GPx-1 and anti-GPx-4 antibodies as the primary antibodies and horseradish peroxidase (HRP)-conjugated antibody as the secondary antibody to determine the expressions of GPx-1 and GPx-4.

Transcriptome analysis of the treated cells was conducted as follows. ZVI sensitive OEC-M1 cells were repeatedly treated with ZVI@CMC NPs at a series of concentrations to obtain ZVI refractory R3 cells. Total RNA of the OEC-M1 and R3 cells was extracted by using the Trizol reagent. Microarray-based transcriptome profiling was performed by using the Human OneArray™ from Phalanx Biotech. The resulting signal segmented files (GPR file) were loaded into the Rosetta Resolver® System for data processing analysis. Standard selection criteria for identifying differentially expressed genes included: (1) fold change log 2≥1 or log 2≤−1, and P<0.05; and (2) log 2 ratio=“NA” and intensity difference between the two samples ≥1000.

In the experimental demonstrations, all animal experiment procedures were approved by the Animal Ethics Committee at National Cheng Kung University. Six to eight week old male mice with severe combined immunodeficiency (SCID) were used as the hosts for OSCC tumor xenografts. 3×10⁶ SAS cells were subcutaneously injected into the dorsal flank of the mice to establish tumor lesions. A visible tumor mass was normally observed 1.5 to 2 weeks after the injection. When the tumor size reached approximately 50 mm³, the mice were randomly assigned to four groups, including a PBS control group, a RSL-3 treatment group, a ZVI@CMC NP treatment group and a ZVI@CMC NP+RSL-3 treatment group (n=4 per group). PBS solution, 20 mg/kg of RSL-3 and 25 mg/kg of ZVI@CMC NPs were individually administered to the mice through tail vein injection once every other day for four times before the final tumor size in every group was measured. The Tumor volumes were measured by a digital caliper and calculated using the formula: 0.5×(length×width).

The quantitative data provided in the experimental demonstrations are presented as means±standard deviation (SD). Statistical analysis was performed with the Prism software (GraphPad). Student's t-test was used to determine the statistical significance (*p<0.05, **p<0.01, ***p<0.001) between the groups; p-values <0.05 were considered significant.

In sum, the methods of using zero valent iron (ZVI) nanoparticles in combination with ferroptosis inducers according to the various embodiments of the present invention provide synergistic therapeutic effect and enhance efficacy of ZVI NP treatments by modulating susceptibility and overcoming resistance of cancer cells to ZVI without affecting normal cells or causing undesirable side effects. Meanwhile, other embodiments of the present invention also reveal the key genetic markers for predicting efficacy of ZVI NP treatment. Therefore, the present invention offers new strategies for treating cancer and improving clinical outcome of nanomedicine.

Previous descriptions are only embodiments of the present disclosure and are not intended to limit the scope of the present disclosure. Many variations and modifications according to the claims and specification of the disclosure are still within the scope of the claimed disclosure. In addition, each of the embodiments and claims does not have to achieve all the advantages or characteristics disclosed. Moreover, the abstract and the title only serve to facilitate searching patent documents and are not intended in any way to limit the scope of the claimed disclosure.

What is claimed is:

1. A method for treating cancer, comprising steps of:
administering to a subject an effective amount of zero valent iron (ZVI) nanoparticles and an effective amount of at least one resistance modulating agent.

2. The method for treating cancer according to claim 1, wherein a shell of each of the ZVI nanoparticles comprises gold (Au).

3. The method for treating cancer according to claim 1, wherein the resistance modulating agent induces ferroptosis, promotes lipid peroxidation, blocks NADP(H) supply, or suppresses metabolism of polyunsaturated fatty acids.

4. The method for treating cancer according to claim 1, wherein the resistance modulating agent suppresses expression of at least one of GSR, AKR1C1, AKR1C3, AKR1B1, AKR1B10, and KYNU genes or promotes expression of at least one of ACSL4, ZEB1 and NNMT genes.

5. The method for treating cancer according to claim 1, wherein the resistance modulating agent is selected from a group consisting of small molecules, peptides, proteins, nucleotides, nanoparticles, and metal-based nanostructures, and
the small molecule comprises erastin, sulfasalazine, sorafenib, buthionine sulfoximine, Ras selective lethal 3 (RSL-3), altretamine, and FIN56.

6. The method for treating cancer according to claim 1, wherein the cancer comprises oral cancer, head and neck cancer, bladder cancer, bone cancer, brain cancer, breast cancer, cervical cancer, colon cancer, endometrial cancer, esophageal cancer, leukemia, liver cancer, lymphoma, kidney cancer, osteosarcoma, ovarian cancer, pancreatic cancer, prostate cancer, skin cancer, small intestine cancer, stomach cancer, thymus cancer and thyroid cancer.

7. A method for improving efficacy of a cancer treatment, comprising steps of:
administering an effective amount of at least one resistance modulating agent to a subject receiving a treatment using zero valent iron (ZVI) nanoparticles.

8. The method for improving efficacy of a cancer treatment according to claim 7, wherein the resistance modulating agent induces ferroptosis, promotes lipid peroxidation, blocks NADP(H) supply, or suppresses metabolism of polyunsaturated fatty acids.

9. The method for improving efficacy of a cancer treatment according to claim 7, wherein the resistance modulating agent suppresses expression of at least one of GSR, AKR1C1, AKR1C3, AKR1B1, AKR1B10 and KYNU genes or promotes expression of at least one of ACSL4, ZEB1 and NNMT genes.

10. The method for improving efficacy of a cancer treatment according to claim 7, wherein the resistance modulating agent is selected from a group consisting of small molecules, peptides, proteins, nucleotides, nanoparticles, and metal-based nanostructures, and
the small molecule comprises erastin, sulfasalazine, sorafenib, buthionine sulfoximine, Ras selective lethal 3 (RSL-3), altretamine, and FIN56.

11. The method for improving efficacy of a cancer treatment according to claim 7, wherein a cancer treated by the cancer treatment comprises oral cancer, head and neck cancer, bladder cancer, bone cancer, brain cancer, breast cancer, cervical cancer, colon cancer, endometrial cancer, esophageal cancer, leukemia, liver cancer, lymphoma, kidney cancer, osteosarcoma, ovarian cancer, pancreatic cancer, prostate cancer, skin cancer, small intestine cancer, stomach cancer, thymus cancer and thyroid cancer.

12. A method for treating cancer, comprising steps of:
obtaining a first transcriptome profile of cancer cells of a subject;
providing the cancer cells a prophylactically effective amount of zero valent iron (ZVI) nanoparticles;
obtaining a second transcriptome profile of the cancer cells;
determining susceptibility of the cancer cells to the ZVI nanoparticles according to a difference between the first transcriptome profile and the second transcriptome profile; and
treating the subject according to a result of the determination.

13. The method for treating cancer according to claim 12, wherein a shell of each of the ZVI nanoparticles comprises gold (Au).

14. The method for treating cancer according to claim 12, wherein the first transcriptome profile and the second transcriptome profile comprise expression levels of at least one of GSR, AKR1C1, AKR1C3, AKR1B1, AKR1B10, KYNU, ACSL4, ZEB1 and NNMT genes.

15. The method for treating cancer according to claim 12, wherein if the cancer cells are determined to be susceptible to the ZVI nanoparticles, the step of treating the subject according to a result of the determination comprises a step of:
administering to the subject a therapeutically effective amount of ZVI nanoparticles.

16. The method for treating cancer according to claim 12, wherein if the cancer cells are determined to be not susceptible to the ZVI nanoparticles, the step of treating the subject according to a result of the determination comprises a step of:
administering to the subject a therapeutically effective amount of ZVI nanoparticles and a therapeutically effective amount of at least one resistance modulating agent.

17. The method for treating cancer according to claim 16, wherein the resistance modulating agent induces ferroptosis, promotes lipid peroxidation, blocks NADP(H) supply, or suppresses metabolism of polyunsaturated fatty acids.

18. The method for treating cancer according to claim 16, wherein the resistance modulating agent suppresses expression of at least one of GSR, AKR1C1, AKR1C3, AKR1B1, AKR1B10 and KYNU genes or promotes expression of at least one of ACSL4, ZEB1 and NNMT genes.

19. The method for treating cancer according to claim 16, wherein the resistance modulating agent is selected from a group consisting of small molecules, peptides, proteins, nucleotides, nanoparticles, and metal-based nanostructures, and
the small molecule comprises erastin, sulfasalazine, sorafenib, buthionine sulfoximine, Ras selective lethal 3 (RSL-3), altretamine, and FIN56.

20. The method for treating cancer according to claim 12, wherein the cancer comprises oral cancer, head and neck cancer, bladder cancer, bone cancer, brain cancer, breast cancer, cervical cancer, colon cancer, endometrial cancer, esophageal cancer, leukemia, liver cancer, lymphoma, kidney cancer, osteosarcoma, ovarian cancer, pancreatic cancer, prostate cancer, skin cancer, small intestine cancer, stomach cancer, thymus cancer and thyroid cancer.
